(19) United States
(12) Patent Application Publication
(10) Pub. No.: US 2004/0024001 A1
(43) Pub. Date: Feb. 5, 2004

(54) Spirobarbituric acid derivatives useful as inhibitors of matrix metalloproteases

(76) Inventors: William J. Pitts, Newtown, PA (US); Soong-Hoon Kim, Titusville, NJ (US); Joseph Barbosa, Lambertville, NJ (US); Wayne Vaccaro, Yardley, PA (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/423,788

(22) Filed: Apr. 25, 2003

Related U.S. Application Data
(60) Provisional application No. 60/375,336, filed on Apr. 25, 2002.
(60) Provisional application No. 60/428,355, filed on Nov. 22, 2002.

(51) Int. Cl. A61K 31/515; C07D 487/10

(52) U.S. Cl. 514/270; 544/231

(57) ABSTRACT

Compound having the formula (I),

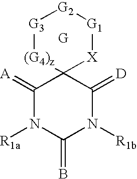

wherein A, B and D are O or S; R₁ₐ and R_{1b} are H, C₁₋₄alkyl, C₂₋₄alkenyl, or C₂₋₄alkynyl; X is —NR₂—, —S—, —S(═O)—, or —S(O)₂—; G₁, G₂ and G₃ are together or separately selected from hetero, carbonyl, alkylene, and alkenylene groups and G₄ is optionally substituted methylene; R₂ is Q-Ar, wherein Q is a linker and Ar is substituted or substituted aryl or heteroaryl; and z is 0 or 1, are useful as inhibitors of MMPs, particularly MMP-13, aggrecanase, and/or TACE.

This application claims priority from U.S. Provisional Applications Ser. Nos. 60/375,336 and 60/428,355 filed Apr. 25, 2002 and Nov. 22, 2002, respectively, incorporated herein by reference in their entirety.

### FIELD OF THE INVENTION

The present invention relates to spiroheterocyclic barbituric acid compounds that inhibit TNF-α converting enzyme (TACE) and metalloproteases (MPs), namely MMP-13 and/or aggrecanase, to pharmaceutical compositions containing these inhibitors, and to the use of these inhibitors in treating diseases associated with cartilage destruction, inflammatory activity, tumor growth and/or metastasis, either alone or in combination with other therapeutic agents.

### BACKGROUND OF THE INVENTION

Many diseases are characterized by the breakdown of connective tissue. For example, loss of cartilage with associated fibrillation is a prominent characteristic of osteoarthritis (OA), and an enhanced rate of cartilage degradation is seen in patients with OA. See Mankin et al., J. Bone Joint Surg. Vol. 52A (1970), at pp 424-434. In severe cases, the cartilage can be degraded to the extent that lesions extend to the bone. Other diseases resulting from connective tissue degradation include rheumatoid arthritis (RA), atherosclerosis, corneal, epidermal or gastric ulceration, and periodontal and bone disease. These diseases are relatively common, particularly in the elderly, and can be extremely painful and debilitating. Additionally, there is evidence that degradation of the extracellular matrix (ECM) facilitates the release and activation of tumor-causing growth factors and is a necessary step in the process of angiogenesis. See Hoekstra et al., Matrix Metalloproteinase Inhibitors: Current Developments and Future Perspectives, The Oncologist, Vol. 5 (2001), at pp. 415-427. Thus, the breakdown of connective tissue is involved in tumor growth and metastasis.

Cartilage is a matrix composed primarily of two proteins, collagen and proteoglycan. Proteoglycan pulls water into tissue and swells against the collagen network, giving cartilage its tensile strength and ability to resist compression. Aggrecan is a major proteoglycan and allows the tissue to bear weight by deforming elastically under pressure. Type II collagen is the major collagen in adult cartilage. Proteoglycan can be readily replaced or repaired, but collagen loss is irreversible. Thus, in normal healthy tissue, there is a strict balance between the production and degradation of collagen. Pathological conditions are characterized by a disruption of that balance and increased proteolysis of collagen. Preventing collagen degradation is thus important to protect against joint destruction, such as that seen in arthritis.

There are four classes of enzymes capable of degrading proteins in mammalian cells: serine, cysteine, aspartic and metalloproteases (MPs). The matrix metalloproteases (MMPs) act extracellularly and can degrade most components of the extracellular matrix (ECM), including collagen and the protein core of proteoglycan. More than 20 different MMPs are now known, which are classified into five subgroups, i.e., collagenases, stromelysins, gelatinases, membrane type, and others. MMP-13 is a collagenase which has a unique ability to effectively cleave type II collagen, although MMP-1 (collagenase-1), MMP-8 (collagenase-2) and MMP-14 are also capable of cleaving type II collagen.

In view of its ability to cleave type II collagen, MMP-13 plays a significant role in cartilage collagen degradation. Current literature suggests that MMP-13 is involved in the pathogenesis of both rheumatoid arthritis (Konttinen et. al, Ann. Rheum. Dis., Vol. 58 [1999], at pp. 691-697) and osteoarthritis (Freemont et al., Ann. Rheum. Dis., Vol. 58 [1999], at pp. 357-365). To illustrate, overexpression of MMP-13 has been observed in osteoarthritic cartilage, and inhibitors targeted to MMP-13 have proven effective at concentrations where MMP-1 (known to cleave type II collagen) was not inhibited. See Mitchell et al., Cloning, Expression, and Type II Collagenolytic Activity of Matrix Metalloproteinase-13 from Human Osteoarthritic Cartilage, J. Clin. Invest., Vol. 93 (3), (1996), at pp. 761-68, and Cawston et al., British J. of Rheumatology, Vol. 37 (1998), at pp. 353-56. Additionally, transgenic mice overexpressing constitutively active human MMP-13 in hyaline cartilage developed osteoarthritic lesions. See Neuhold et al., J. Clin. Invest., Vol. 107 (2001), at pp. 35-44.

Normally, the MP enzymes are tightly regulated at the level of their synthesis and also at the extracellular level through the action of specific inhibitors, such as alpha-2-macroglobulins and TIMPs (tissue inhibitors of metalloprotease), which form inactive complexes with the MPs. This regulation of MP enzymes promotes the strict balance of collagen production and degradation, referenced above, to maintain healthy connective tissue. However, under pathophysiological conditions, the mechanisms for regulating MPs are disturbed, giving rise to diseases such as those referenced above. Additionally, increased MMP expression (or an imbalance in favor of MMP activity) plays a role in pathophysiology of cancer in that the breakdown of the ECM enables the invasion of tumor cells through the ECM. Animal tumor models evidence that MMPs play a role in the process of mestastasis. See Hoekstra et al., cited above.

While MMP-13 is involved with collagen modulation, aggrecanase is an MP that is capable of providing the specific cleavage product of proteoglycan that has been found in RA and OA patients. See Lohmander et al., Arthritis Rheum., Vol. 36 (1993), at pp. 1214-22). Thus, inhibition of aggrecanase may provide some benefit in arthritic diseases. Furthermore, the enzyme active site of MMPs, particularly MMP-13, shares common features with the enzyme active site of TNF-α converting enzyme (TACE), the enzyme responsible for TNF-α release from cells. TACE was recently purified and sequenced (Black et al., Nature, Vol. 385 [1997], at p. 729; Moss et al., Nature, Vol. 385 [1997], at p. 733), and it is believed its inhibition is effective in controlling excessive TNF-α production. There is considerable evidence that inhibiting TNF-α is beneficial for treating many inflammatory and autoimmune diseases, such as rheumatoid arthritis, non-insulin dependent diabetes melitus, and Crohn's disease. Notably, excessive TNF-α production has been observed in several disease conditions also characterized by MMP-mediated tissue degradation, confirming a correlation between TNF-α and/or TACE and MMPs. Accordingly, compounds that inhibit both MMPs and TNF-α production may have a particular advantage in diseases where both mechanisms are involved. Alternatively, co-administration of an MMP inhibitor and TACE inhibitor would be expected to be beneficial.

The instant invention provides spiroheterocyclic barbituric acid compounds that act as inhibitors of MMPs, in particular MMP-13 and/or aggrecanase. Thus, they are useful as cartilage protecting therapeutics, in treating diseases such as RA and OA, and also as angiogenesis inhibitors, in treating tumors and metastases. Additionally, the compounds of this invention inhibit TACE and are thus useful in treating inflammatory and immunosuppressive diseases. Spirobarbituric compounds are disclosed in Kokel et al., Bull. Soc. Chim. Belg., Vol. 106(5) (1997), at pp. 293-94; Aly et al., Tetrahedron, Vol. 50(3) (1994), at pp. 895-906; Kato et al., J. Electroanal. Chem. Interfacial Electrochem., Vol. 80(1) (1977), at pp. 181-99; Senda et al., Yakugaku Zasshi, “Pyrimidine deriviatives and related compounds, Synthesis of bucolome related compounds,” Vol. 91(12) (1971), at pp. 1367-71; Moore et al., titled “Synthesis of 6-hydroxy-1,3,4,5-tetrahydrobenz[c,d]indole,” which discloses the compound 1,7,9-Triazaspiro[4,5]decane-2,6,8,10-tetrone, 3 methyl; U.S. Pat. No. 4,049,698 to Eastman Kodak Co.; U.S. Pat. Nos. 3,270,019 and 3,247,205 to Minnesota Mining and Mfg. Co.; and JP Patent No. 2001089479 to Fuji Photo Film Co., Ltd., titled “Preparation of pyrrolinoazabenzenes as dyes.”

### SUMMARY OF THE INVENTION

The present invention provides heterocyclic compounds of the following formula

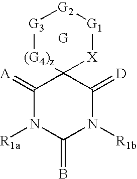

or a pharmaceutically-acceptable salt, hydrate, or prodrug thereof, wherein:

A, B and D are independently selected from oxygen and sulfur;

one of R₁ₐ and R_{1b} is hydrogen and the other of R₁ₐ and R_{1b} is selected from hydrogen, C₁₋₄alkyl, C₂₋₄alkenyl, and C₂₋₄alkynyl;

X is —NR₂—, —S—, —S(═O)—, or —S(O)₂—;

G₁ is —C(═O)—, —CR₃R₄—, —NR₁₁—, —CR₄═, or —N═;

G₂ is —O—, —C(═O)—, —CR₅R₆—, —NR₁₂—, —N═, or —CR₅═, except when G₁ is —CR₄═, or —N═, then G₂ is ═N— or ═CR₅—;

G₃ is —CR₇R₈—, —NR₁₃—, —N═, or —CR₇═, except when G₂ is —CR₅═ or —N═, then G₃ is ═CR₇— or ═N—;

G₄ is —CR₉R₁₀—, except when G₃ is —CR₇═ or —N═, then G₄ is ═CR₉—;

R₂ is Q-Ar, wherein Ar is substituted or unsubstituted aryl or substituted or unsubstituted heteroaryl, and Q is —C(═O)—, —CHR₁₄—, or a bond;

is R₃, R₄, R₅, R₆, R₇, R₈, R₉, and R₁₀ are independently selected from the group consisting of hydrogen, halogen, nitro, cyano, alkyl, substituted alkyl, alkenyl, substituted alkenyl, hydroxy, alkoxy, phenyloxy, benzyloxy, amino, alkylamino, C(═O)H, acyl, CO₂H, alkoxycarbonyl, carbamyl, alkylthio, sulfonyl, sulfonamidyl, cycloalkyl, heterocycle, aryl, and heteroaryl, wherein each of R₃, R₄, R₅, R₆, R₇, R₈, R₉, and R₁₀ in turn is optionally substituted with one to two substituents selected from R₁₅;

R₁₁, R₁₂, and R₁₃ are independently selected from hydrogen, cyano, alkyl, substituted alkyl, alkenyl, substituted alkenyl, hydroxy, alkoxy, phenyloxy, benzyloxy, alkylamino, —C(═O)H, acyl, —CO₂H, alkoxycarbonyl, carbamyl, sulfonyl, sulfonamidyl, cycloalkyl, heterocycle, aryl, and heteroaryl, wherein each of R₁₁, R₁₂, and R₁₃ in turn is optionally substituted with one to two substituents selected from R₁₆;

R₁₄ is hydrogen, halogen, C₁₋₄alkyl, OH, OCH₃, or NH₂;

R₁₅ and R₁₆ are at each occurrence selected independently of each other from C₁₋₄alkyl, halogen, nitro, cyano, hydroxy, haloC₁₋₄alkyl, haloC₁₋₄alkoxy, amino, C₁₋₄alkylamino, C₁₋₄aminoalkyl, C₁₋₄hydroxyalkyl, C₃₋₇cycloalkyl, four to seven membered heterocyclo, five or six membered heteroaryl, phenyl, benzyl, phenyloxy, and benzyloxy; and

z is 0, 1, or 2 so that ring G is a four-to-seven membered spiroheterocyclo ring;

provided that when Q is a bond,

(a) G2 are selected from —O— and C(═O); or

(b) Ar is aryl or heteroaryl, each group optionally substituted with one to three of R₁₈ wherein:

R₁₈ is selected from alkyl, halogen, nitro, cyano, haloalkyl, haloalkoxy, hydroxy, alkoxy, (>C₁₀)aryl, (>C₈)cycloalkyl, (>C₁₀)heterocyclo, (>C₁₀)heteroaryl, A₁-NH-A₂-R₂₅, -A₁-O-A₂-R₂₆, -A₁-OC(═O)-A₂-R₂₅, -A₁-CO₂-A₂-R₂₅, -A₁-NR₁₉C(═O)-A₂-R₂₅, -A₁-NR₁₉C(═O)NR₂₀-A₂-R₂₅, -A₁-NR₁₉CO₂-A₂-R₂₅, -A₁-NR₁₉SO₂NR₂₀-A₂-R₂₅, -A₁-C(═O)NR₁₉-A₂-R₂₅, -A₃-O-A₂, A₃-S-A₂, -A₃-SO₂-A₂-, -A₃-NR₁₉-A₂-, -A4-C(═O)-A₅-, A₄-S(═O)-A₅-, -A₄-NR₁₉SO₂-A₅-, and -A₄-SO₂NR₁₉-A₅-,

A₁ is —(CR₂₁R₂₂)ᵣ—;

A₂ is —(CR₂₃R₂₄)ₛ—;

A₃ is —(CR₂₁R₂₂)ₜ—;

A₄ is —(CR₂₁R₂₂)ᵤ—;

A₅ is —(CR₂₃R₂₄)ᵥ—;

r and s are selected from 0, 1, 2, 3, and 4;

t is 2, 3 or 4;

u and v are 0-4 provided that u and v are not both 0;

R₁₉, R₂₀, R₂₁, R₂₂, R₂₃, and R₂₄ are selected from hydrogen, C₁₋₄alkyl, hydroxyC₁₋₄alkyl, haloC₁₋₄alkyl, amino, and aminoC₁₋₄alkyl;

R₂₅ is selected from hydrogen, C₁₋₆alkyl, amino, C₁₋₆alkylamino, aryl, cycloalkyl, heterocyclo, and heteroaryl, each group optionally substituted with R₂₇ and/or R₂₈;

R₂₆ is

(a) diphenoxy, (>C₈)cycloalkyl, (>C₁₀)heterocyclo, or (>C₁₀)heteroaryl, each group optionally substituted with R₂₇ and/or R₂₈; or

(b) aryl substituted with —SC₁₋₄alkyl, —OC₁₋₄alkyl, —SC₁₋₄haloalkyl, —OC₁₋₄haloalkyl, nitro, haloalkyl, C₂₋₄alkenyl, —CO₂H, —CO₂C₁₋₄alkyl, —SO₂C₁₋₄alkyl or —C(═O) C₁₋₄alkyl; and

R₂₇ and R₂₈ are independently selected from C₁₋₄alkyl, C₂₋₄alkenyl, hydroxy, —OC₁₋₄alkyl, halogen, cyano, nitro, —CF₃, —OC₁₋₄haloalkyl, —SC₁₋₄alkyl, —SO₂C₁₋₄alkyl, —CO₂H, —CO₂C₁₋₄alkyl, —C(═O) C₁₋₄alkyl, phenyloxy, and benzyloxy.

The invention further relates to pharmaceutical compositions including one or more compounds according to formula (I), and to methods of inhibiting metalloproteases such as MMP-13 and/or aggrecanase, and/or TACE, comprising administering an effective amount of at least one compound of formula (I) to a patient in need thereof.

### DETAILED DESCRIPTION OF THE INVENTION

The following are definitions of the terms as used throughout this specification and claims. The initial definition provided for a group or term herein applies to that group or term throughout the present specification, individually or as part of another group, unless otherwise indicated.

The term “alkyl” refers to straight or branched chain hydrocarbon groups having 1 to 12 carbon atoms, preferably 1 to 8 carbon atoms, such as methyl, ethyl, n-propyl, i-propyl, n-butyl, i-butyl, t-butyl, pentyl, hexyl, heptyl, octyl, etc. Lower alkyl groups, that is, alkyl groups of 1 to 4 carbon atoms, are most preferred.

The term “substituted alkyl” refers to alkyl groups substituted with one, two or three groups selected from halo, cyano, keto (═O), ORₐ, SRₐ, NRₐR_{b}, (C═O)Rₐ, —CO₂Rₐ, C(═O)NRₐR_{b}, NRₐC(═O)R_{b}, NRₐCO₂R_{b}, —OC(═O)Rₐ, —OC(═O)NRₐR_{b}, —NR_{c}C(═O)NRₐR_{b}, NRₐSO₂R_{d}, SO₂R_{d}, SO₃R_{d}, cycloalkyl, substituted cycloalkyl, aryl, substituted aryl, heteroaryl, substituted heteroaryl, heterocycle, and substituted heterocyclo, wherein the groups Rₐ, R_{b}, and R_{c} are selected from hydrogen, C₁₋₆alkyl, cycloalkyl, substituted cycloalkyl, aryl, substituted aryl, heteroaryl, substituted heteroaryl, heterocycle, substituted heterocyclo, or C₁₋₆alkyl substituted with one, two or three of halogen, hydroxy, O(alkyl), O(phenyl), O(benzyl), nitro, amino, cyano, —(C═O)H, —CO₂H, —(C═O)alkyl, —CO₂alkyl, —NH(alkyl), —NH(cycloalkyl), —NH(aryl), —NH(heterocyclo), —N(alkyl)₂, —C(═O)H, acyl, —C(═O)phenyl, carboxy, —CO₂-alkyl, —(C═O)NH₂, —(C═O)NH(alkyl), —(C═O)NH(cycloalkyl), —(C═O)N(alkyl)₂, —C(═O)—(CH₂)₁₋₂NH₂, —C(═O)—(CH₂)₁₋₂NH(alkyl), —C(═O)—(CH₂)₁₋₂N(alkyl)₂, —NH—CH₂-carboxy, —NH—CH₂-CO₂-alkyl, phenyl, benzyl, C₃₋₇cycloalkyl, four to seven membered heterocyclo, and/or five or six membered heteroaryl. The group R_{d} may be selected from the same groups as Rₐ, R_{b} and R_{c} but is not hydrogen. Alternatively, the groups Rₐ and R_{b} may together form a heterocyclo or heteroaryl ring. It should be understood that when a substituted alkyl group is substituted with a substituted aryl, cycloalkyl, heteroaryl, or heterocyclo, such rings are as defined below and thus may have one to three substituents as set forth below in the definitions for these terms.

When the term “alkyl” is used as a suffix following another specifically-named group, e.g., arylalkyl, heteroarylalkyl, hydroxyalkyl, the term defines with more specificity at least one of the substituents that the substituted alkyl will contain. For example, arylalkyl refers to an aryl bonded through an alkyl, or in other words, a substituted alkyl group having from 1 to 12 carbon atoms and at least one substituent that is aryl (e.g., benzyl or biphenyl). “Lower arylalkyl” refers to substituted alkyl groups having 1 to 4 carbon atoms and at least one aryl substituent.

When a subscript is used in conjunction with a group such as C₁₋₄alkyl, the subscript refers to the number of carbon atoms that the group will contain, in addition to heteroatoms. Thus, the term hydroxyC₁₋₄alkyl or C₁₋₄hydroxyalkyl refers to an alkyl group of one to four carbon atoms having an OH substituent on one of the carbon atoms. The term C₁₋₂alkylamino refers to an alkylamino group having one or two carbon atoms, i.e., NHCH₃, N(CH₃)₂, or NHCH₂CH₃.

Also, where the symbol “>” is used in a group the symbol “>” is intended to mean “greater than”. For example, the group “(>C₁₀)aryl” designates an aryl group having greater than ten carbon atoms, in addition to optional heteroatoms.

The term “alkenyl” refers to straight or branched chain hydrocarbon groups having 2 to 12 carbon atoms and at least one double bond. Alkenyl groups of 2 to 6 carbon atoms and having one double bond are most preferred.

The term “alkynyl” refers to straight or branched chain hydrocarbon groups having 2 to 12 carbon atoms and at least one triple bond. Alkynyl groups of 2 to 6 carbon atoms and having one triple bond are most preferred. A substituted alkenyl or alkynyl will contain one, two, or three substituents as defined above for alkyl groups.

The term “alkylene” refers to bivalent straight or branched chain hydrocarbon groups having 1 to 12 carbon atoms, preferably 1 to 8 carbon atoms, e.g., {—CH₂—}ₙ, wherein n is 1 to 12, preferably 1-8. Lower alkylene groups, that is, alkylene groups of 1 to 4 carbon atoms, are most preferred. The terms “alkenylene” and “alkynylene” refer to bivalent radicals of alkenyl and alknyl groups, respectively, as defined above. Substituted alkylene, alkenylene, and alkynylene groups may have substituents as defined above for the monovalent groups.

The term “alkoxy” refers to the group ORₑ wherein Rₑ is alkyl, alkenyl, alkynyl, heterocycle, or cycloalkyl. Thus, an alkoxy includes such groups as methoxy, ethoxy, cyclopropyloxy, pyrrolidinyloxy, and so forth. The term “aryloxy” refers to the groups O(aryl) or O(heteroaryl), wherein aryl and heteroaryl are as defined below. A substituted alkoxy or aryloxy will have one, two or three substituents as defined herein for the respective alkyl or aryl group.

The term “alkylthio” refers to an alkyl or substituted alkyl group as defined above bonded through one or more sulfur (—S—) atoms, e.g., —S(alkyl) or —S(substituted alkyl).

The term “alkylamino” refers to the groups NHR and NRR, wherein R is alkyl or substituted alkyl as defined above.

The term “acyl” refers to an alkyl or substituted alkyl group as defined above bonded through one or more carbonyl {—C(═O)—) groups. When the term acyl is used in conjunction with another group, as in acylamino, this refers to the carbonyl group {—C(═O)} linked to the second named group. Thus, for example, acylamino refers to —C(═O)NH₂ and acylaryl refers to —C(═O)(aryl).

The term “halo” or “halogen” refers to chloro, bromo, fluoro and iodo.

The term carbamyl refers to the group C(═O)NR_{f}R_{g} wherein R_{f} and R_{g} may be, selected from hydrogen, alkyl, and substituted alkyl.

The term “carboxy” when used alone refers to the group CO₂H. “Carboxyalkyl”refers to the group CO₂R, wherein R is alkyl or substituted alkyl, as defined above.

The term “sulfonamide” or “sulfonamidyl” refers to the group —S(O)₂NR_{f}R_{g}, wherein R_{f} and R_{g} are as defined above for carbamyl.

The term “sulphonyl” or “sulfonyl” refers to the group —S(O)₁₋₂—R, wherein R is alkyl or substituted alkyl, as defined above.

The term “cycloalkyl” refers to monocyclic or bicyclic hydrocarbon groups of 3 to 9 carbon atoms which are, respectively, fully saturated or partially unsaturated. The term “cycloalkyl” includes such saturated or partially unsaturated carbocyclic rings having a carbon-carbon bridge of three to four carbon atoms or having 1 or 2 aromatic or heterocyclo rings fused thereto. Thus, the term “cycloalkyl” includes cyclopropyl, cyclobutyl, cyclopentyl, cyclohexyl, cycloheptyl, cyclooctyl, etc., as well as

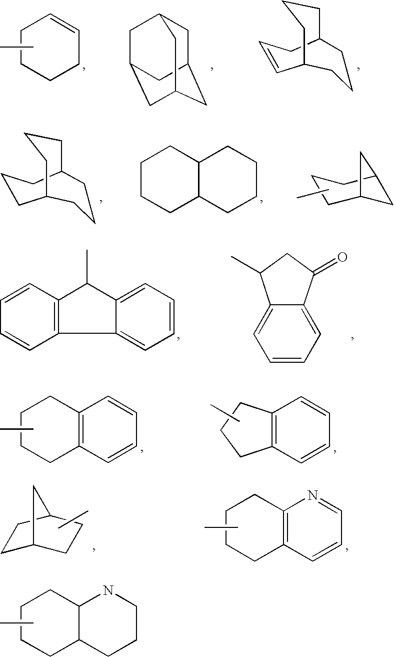

and the like.

The term “substituted cycloalkyl” unless otherwise indicated includes cycloalkyl groups as defined above substituted with one, two or three groups selected from (i) Rh, (ii) keto (═O), and/or (iii) C₁₋₄alkyl or C₂₋₆alkenyl optionally substituted with one to three of Rₕ, wherein Rₕ is halogen, nitro, cyano, haloalkyl, haloalkoxy, aryl, cycloalkyl, heterocyclo, heteroaryl, —X₁—O—X₂—Rᵢ, —X₁—S—X₂—Rᵢ, —X₁—C(═O)—X₂—Rᵢ, —X₁—OC(═O)—X₂—Rᵢ, —X₁—S(═O)—X₂—Rᵢ, —X₁—SO₂—X₂—Rᵢ, —X₁—CO₂—X₂—Rᵢ, —X₁—NRⱼ—X₂—Rᵢ, —X₁—NRⱼC(═O)—X₂—Rᵢ, —X₁—NRⱼC(═O)NRₖ—X₂—Rᵢ, —X₁—NRⱼCO₂—X₂—Rᵢ, —X₁—NRⱼSO₂—X₂—Rᵢ, —X₁—NRⱼSO₂NRₖ—X₂—Rᵢ, —X₁—SO₂NRⱼ—X₂—Rᵢ, or —X₁—C(═O)NRⱼ—X₂—Rᵢ; wherein X₁ and X₂ are —(CRⱼRₖ)_{w}—; Rⱼ and Rₖ are selected from hydrogen, alkyl, hydroxyalkyl, haloalkyl, amino, and aminoalkyl; Rᵢ is selected from hydrogen, alkyl, amino, alkylamino, aryl, cycloalkyl, heterocyclo, and heteroaryl; and w is 0 to 6; wherein each Rᵢ in turn is optionally substituted with one to two of C₁₋₄alkyl, C₂₋₄alkenyl, —O(C₁₋₄alkyl), halogen, cyano, nitro, —C₁₋₄haloalkyl, —O(C₁₋₄haloalkyl), —S(C₁₋₄alkyl), —SO₂(C₁₋₄alkyl), —CO₂H, —CO₂(C₁₋₄alkyl), —C(═O)H, —C(═O)(C₁₋₄alkyl), —NH₂, —NH(C₁₋₆alkyl), —N(C₁₋₆alkyl)₂, phenyloxy, benzyloxy, and/or lower alkyl substituted with one to two hydroxy, halogen, cyano, —O(C₁₋₄alkyl), —O(C₂₋₄alkenyl), amino, C₁₋₄alkylamino, halogen, cyano, nitro, trifluoromethyl, trifluoromethoxy, nitro, —S(C₁₋₄alkyl), —SO₂C₁₋₄alkyl, —CO₂H, —CO₂(C₁₋₄alkyl), —C(═O)H, and/or —C(═O)(C₁₋₄alkyl).

The term “aryl” refers to phenyl, 1-naphthyl and 2-naphthyl, with phenyl being preferred, as well as such rings having fused thereto a cycloalkyl, cycloalkenyl, heterocyclo, or heteroaryl ring. Examples include:

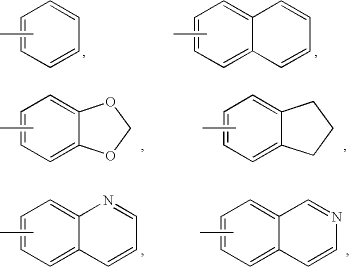

and the like.

The term “substituted aryl” includes such rings having one, two or three substituents selected from (i) Rₕ and (ii) C₁₋₆alkyl or C₂₋₆alkenyl optionally substituted lo with one to two of Rₕ and/or keto (═O), wherein Rₕ is as defined above for cycloalkyl and has the various optional substituents as defined above for cycloalkyl. The term “substituted phenyl” means a phenyl group having one, two or three substituents as defined for aryl.

The term “diphenoxy” refers to the structure:

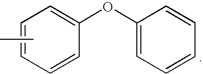

The term “substituted diphenoxy” includes the structure immediately above having one, two or three substituents selected from (i) Rₕ and (ii) C₁₋₆alkyl or C₂₋₆alkenyl optionally substituted with one to two of Rₕ and/or keto (═O), wherein Rₕ is as defined above for cycloalkyl and has the various optional substituents as defined above for cycloalkyl. The term “substituted diphenoxy” means a diphenoxy group having one, two or three substituents as defined for aryl.

The term “carbocyclo” or “carbocyclic” refers to a cyclic group in which all ring atoms are carbon, including substituted or unsubstituted cycloalkyl and aryl groups, as defined herein.

The term “heterocyclo” or “heterocycle” refers to non-aromatic 3 to 7 membered monocyclic groups, 7 to 11 membered bicyclic groups, and 10 to 15 membered tricyclic groups which have at least one heteroatom (O, S or N) in at least one of the rings. Each ring of the heterocyclo group containing a heteroatom can contain one or two oxygen or sulfur atoms and/or from one to four nitrogen atoms provided that the total number of heteroatoms in each ring is four or less, and further provided that the ring contains at least one carbon atom. The rings completing the bicyclic and tricyclic groups may contain only carbon atoms and may be saturated, partially saturated, or unsaturated, and they may be either fused, bridged, and/or joined through one or more spiro unions. The nitrogen and sulfur atoms may optionally be oxidized and the nitrogen atoms may optionally be quaternized. The heterocyclo group may be attached at any available nitrogen or carbon atom. Exemplary heterocyclic groups include

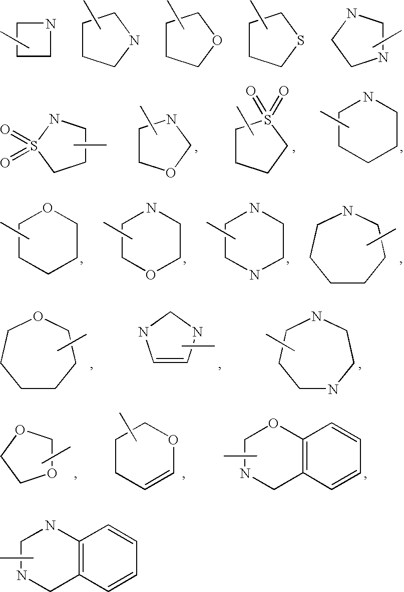

and the like.

The term “substituted heterocyclo” refers to a heterocyclo ring as defined containing one, two or three substituents at any available carbon or nitrogen atom selected from Rₕ, keto (═O), and C₁₋₆alkyl or C₂₋₆alkenyl optionally substituted with one to two of Rₕ and/or keto (═O), wherein Rₕ is as defined above for cycloalkyl and has the various optional substituents as defined above for cycloalkyl.

The term “heteroaryl” refers to aromatic 5 or 6 membered monocyclic groups, 9 or 10 membered bicyclic groups, and 11 to 14 membered tricyclic groups which have at least one heteroatom (O, S or N) in at least one of the rings. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic and tricyclic groups may contain only carbon atoms and may be saturated, partially saturated, or unsaturated. The nitrogen and sulfur atoms may optionally be oxidized and the nitrogen atoms may optionally be quaternized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring. Examples of heteroaryl rings include

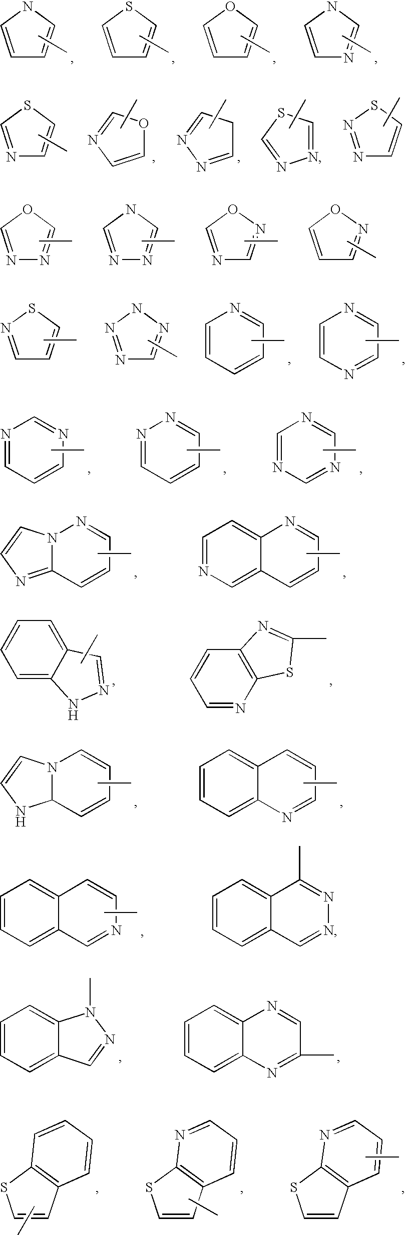

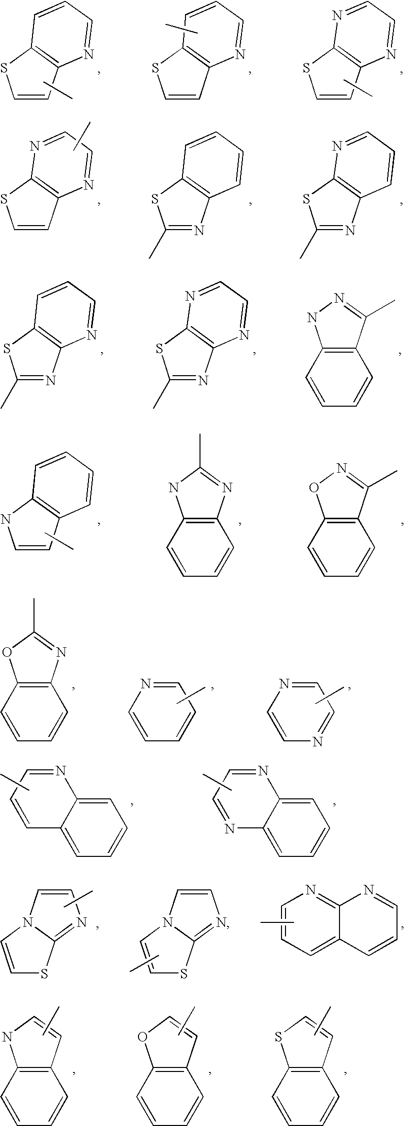

and the like.

The term “substituted heteroaryl” refers to a heteroaryl ring as defined containing one, two or three substituents at any available carbon or nitrogen atom selected from Rₕ, and C₁₋₆alkyl or C₂₋₆alkenyl optionally substituted with one to two of Rₕ and/or keto (═O), wherein Rₕ is as defined above for cycloalkyl and has the various optional substituents as defined above for cycloalkyl.

When reference is made herein to a particularly-named substituted aryl, cycloalkyl, heterocyclic or heteroaryl group, such as diphenoxy, imidazolyl, piperazinyl, and so forth, the named ring may optionally contain one or more (preferably one to three) substituents selected from the substituents recited above for heteroaryl and heterocyclo groups, as appropriate. Thus, the term substituted phenyl means a phenyl group having one, two or three substituents as defined above for aryl.

The term “heteroatoms” shall include oxygen, sulfur and nitrogen.

The term “haloalkyl” means an alkyl having one or more halo substituents.

The term “haloalkoxy” means an alkoxy group having one or more halo substituents. For example, “haloalkoxy” includes —OCF₃.

When the term “unsaturated” is used herein to refer to a ring or group, the ring or group may be fully unsaturated or partially unsaturated.

The phrase “optionally substituted” is intended to be synonymous with “substituted or unsubstituted”. Accordingly, an “optionally substituted diphenoxy” may be either substituted or unsubstituted.

Throughout the specification, groups and substituents thereof may be chosen by one skilled in the field to provide stable moieties and compounds.

The compounds of Formula (I) may form salts with alkali metals such as sodium, potassium and lithium, with alkaline earth metals such as calcium and magnesium, with organic bases such as dicyclohexylamine, tributylamine, pyridine and amino acids such as arginine, lysine and the like. The compounds for Formula (I) may form salts with a variety of organic and inorganic acids. Such salts include those formed with hydrogen chloride, hydrogen bromide, methanesulfonic acid, sulfuric acid, acetic acid, trifluoroacetic acid, oxalic acid, maleic acid, benzenesulfonic acid, toluenesulfonic acid and various others (e.g., nitrates, phosphates, borates, tartrates, citrates, succinates, benzoates, ascorbates, salicylates and the like). Such salts can be formed as known to those skilled in the art. Salt forms of the compounds may be advantageous for improving the compound dissolution rate and oral bioavailability. Pharmaceutically-acceptable salts are preferred although other salts may be useful, e.g., as intermediates to prepared pharmaceutically-acceptable salts.

In addition, zwitterions (“inner salts”) may be formed.

All stereoisomers of the compounds of the instant invention are contemplated, either in admixture or in pure or substantially pure form. The definition of compounds according to the invention embraces all the possible stereoisomers and their mixtures. It embraces the racemic forms and the isolated optical isomers having the specified activity. The racemic forms can be resolved by physical methods, such as, for example, fractional crystallization, separation or crystallization of diastereomeric derivatives or separation by chiral column chromatography. The individual optical isomers can be obtained from the racemates from the conventional methods, such as, for example, salt formation with an optically active acid followed by crystallization.

Compounds of the Formula (I) may also have prodrug forms. Any compound that will be converted in vivo to provide the bioactive agent (i.e., a compound of formula I) is a prodrug within the scope and spirit of the invention.

Various forms of prodrugs are well known in the art. For examples of such prodrug derivatives, see:

a) Design of Prodrugs, edited by H. Bundgaard, (Elsevier, 1985) and Methods in Enzymology, Vol.42, p. 309-396, edited by K. Widder, et al. (Acamedic Press, 1985);

b) A Textbook of Drug Design and Development, edited by Krosgaard-Larsen and H. Bundgaard, Chapter 5, “Design and Application of Prodrugs,” by H. Bundgaard, pp. 113-191 (1991); and

c) H. Bundgaard, Advanced Drug Delivery Reviews, 8, 1-38 (1992), each of which is incorporated herein by reference.

It should further be understood that solvates (e.g., hydrates) of the compounds of Formula (I) are also with the scope of the present invention. Methods of solvation are generally known in the art.

Multiple substituents may be selected for any compound within the scope of this invention; however, advantageously substituents are selected so that the compounds of formula (I) have a molecular weight of less than 1,500. More preferred are compounds having a molecular weight of less than 1,000, and even more preferred are compounds having a molecular of less than 500.

Preferred Compounds

Preferred compounds are those having the formula (Ia),

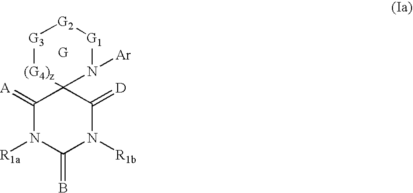

or a pharmaceutically-acceptable salt, hydrate, or prodrug thereof, in which:

one of R₁ₐ and R_{1b} is hydrogen and the other of R₁ₐ and R_{1b} is selected from hydrogen, C₁₋₄alkyl, C₂₋₄alkenyl, and C₂₋₄alkynyl;

X is —NR₂—, —S—, —S(═O)—, or —S(O)₂—;

G₁ is —C(═O)—, —CR₃R₄—, —NR₁₁—, —CR₄═, or —N═;

G₂ is —O—, —C(═O)—, —CR₅R₆—, —NR₁₂—, —N═, or —CR₅═, except when G₁ is —CR₄═, or —N═, then G₂ is ═N— or ═CR₅—;

G₃ is —CR₇R₈—, —NR₁₃—, —N═, or —CR₇═, except when G₂ is —CR₅═ or —N═, then G₃ is ═CR₇— or ═N—;

G₄ is —CR₉R₁₀—, except when G₃ is —CR₇═ or —N═, then G₄ is ═CR₉—;

Ar is aryl or heteroaryl, each group optionally substituted with one to two R₁₈;

R₃, R₄, R₅, R₆, R₇, R₈, R₉, and R₁₀ are independently selected from the group consisting of hydrogen, halogen, nitro, cyano, C₁₋₄alkyl, hydroxy, trifluoromethyl, trifluoromethoxy, N-phenyloxy, benzyloxy, alkylamino, C(═O)H, CO₂H, C(═O) (C₁₋₄alkyl), and CO₂(C₁₋₄alkyl);

R₁₁ and R₁₂ are independently hydrogen or C₁₋₄alkyl;

R₁₈ is selected from alkyl, halogen, nitro, cyano, haloalkyl, haloalkoxy, hydroxy, alkoxy, (>C₁₀)aryl, (>C₈)cycloalkyl, (>C₁₀)heterocyclo, (>C₁₀)heteroaryl, A₁-NH-A₂-R₂₅, -A₁-O-A₂-R₂₆, -A₁-OC(═O)-A₂-R₂₅, -A₁-CO₂-A₂-R₂₅, -A₁-NR₁₉C(═O)-A₂-R₂₅, -A₁-NR₁₉C(═O)NR₂₀-A₂-R₂₅, -A₁-NR₁₉CO₂-A₂-R₂₅, -A₁-NR₁₉SO₂NR₂₀-A₂-R₂₅, -A₁-C(═O)NR₁₉-A₂-R₂₅, -A₃-O-A₂, A₃-S-A₂, -A₃-SO₂-A₂-, -A₃-NR₁₉-A₂-, -A₄-C(═O)-A₅-, A₄-S(═O)-A₅-, -A₄-NR₁₉SO₂-A₅-, and -A₄-SO₂NR₁₉-A₅-;

A₁ is —(CR₂₁R₂₂)ᵣ—;

A₂ is —(CR₂₃R₂₄)ₛ—;

A₃ is —(CR₂₁R₂₂)ₜ—;

A₄ is —(CR₂₁R₂₂)ᵤ—;

A₅ is —(CR₂₃R₂₄)ᵥ—;

r and s are selected from 0, 1, 2, 3, and 4;

t is 2, 3 or 4;

u and v are 0-4 provided that u and v are not both 0;

R₁₉, R₂₀, R₂₁, R₂₂, R₂₃, and R₂₄ are selected from hydrogen, C₁₋₄alkyl, hydroxyC₁₋₄alkyl haloC₁₋₄alkyl, amino, and aminoC₁₋₄alkyl;

R₂₅ is selected from hydrogen, C₁₋₆alkyl, amino, C₁₋₆alkylamino, aryl, cycloalkyl, heterocyclo, and heteroaryl, each group optionally substituted with R₂₇ and/or R₂₈;

R₂₆ is

(a) diphenoxy, (>C₈)cycloalkyl, (>C₁₀)heterocyclo, or (>C₁₀)heteroaryl, each group optionally substituted with R₂₇ and/or R₂₈;or

(b) aryl substituted with —SC₁₋₄alkyl, —OC₁₋₄alkyl, —SC₁₋₄haloalkyl, —OC₁₋₄haloalkyl, nitro, haloalkyl, C₂₀₄alkenyl, —CO₂H, —CO₂C₁₋₄alkyl, —SO₂C₁₋₄alkyl or —C(═O) C₁₋₄alkyl; and

R₂₇ and R₂₈ are independently selected from C₁₋₄alkyl, C₂₋₄alkenyl, hydroxy, —OC₁₋₄alkyl, halogen, cyano, nitro, —CF₃, —OC₁₋₄haloalkyl, —SC₁₋₄alkyl, —SO₂C₁₋₄alkyl, —CO₂H, —CO₂C₁₋₄alkyl, —C(═O) C₁₋₄alkyl, phenyloxy, and benzyloxy; and

z is 0, or 1 so that ring G is a five to six-membered spiroheterocyclo ring.

Preferably, ring G is selected from:

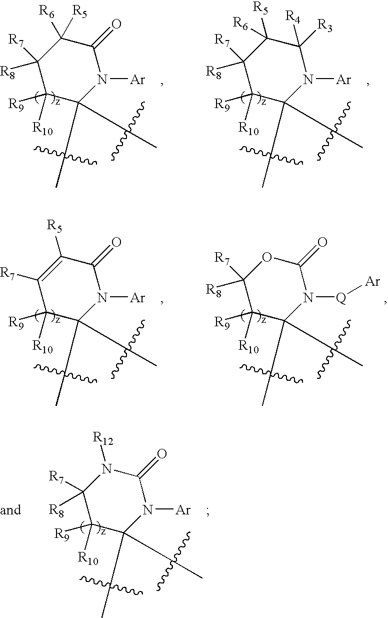

in which

R₃, R₄, R₅, R₆, R₇, R₈, R₉, and R₁₀ are independently selected from the group consisting of hydrogen, halogen, nitro, cyano, amino, C₁₋₄alkyl, C₂₋₄alkenyl, hydroxy, C₁₋₄alkoxy, phenyloxy, benzyloxy, C₁₋₄alkylamino, C₁₋₄aminoalkyl, C₁₋₄hydroxyalkyl, C₃₋₇cycloalkyl, four to seven membered heterocyclo, five or six membered heteroaryl, phenyl, benzyl, phenyloxy, and benzyloxy;

R₁₄ is hydrogen, halogen, C₁₋₄alkyl, OH, OCH₃, or NH₂;

r and s are independently selected from 0, 1, and 2; and

z is 0 or 1.

More preferred are compounds having the formula (Ib)

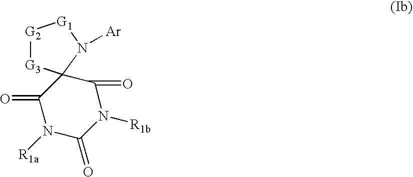

or a pharmaceutically-acceptable salt, hydrate or prodrug thereof, in which:

one of G₁ and G₂ is CR₅R₆, and the other of G₁ and G₂ is —C(═O)—;

G₃ is —CR₇R₈;

R₅, R₆, R₇ and R₈ are independently H or alkyl; and

Ar is phenyl, pyridyl, pyrazinyl, pyrimidinyl, or napthyl, each of which is optionally substituted with R₁₈;

R₁₈ is selected from C₁₋₄alkyl, halogen, hydroxy, C₁₋₄alkyoxy, A₁-NH-A₂-R₂₅, -O-A₁-NHC(═O)R₂₅, —O—R₂₆ or (>C10)heteroaryl;

R₂₅ is selected from hydrogen, C₁₋₆alkyl, phenyl, pyridyl, indolyl, napthyl and

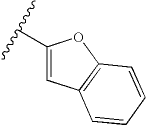

each R₂₅ optionally substituted with R₂₇ and/or R₂₈ where valence allows;

R₂₆ is

(a) diphenoxy or (>C₁₀)heteroaryl, each group optionally substituted with R₂₇ and/or R₂₈; or

(b) phenyl substituted with —SCH₃, —O CH₃, —S CF₃, —OC₁₋₄ CF₃, nitro, CF₃, C₂alkenyl, —CO₂H, —CO₂ CH₃, —SO₂ CH₃ or —C(═O)CH₃; and

R₂₇ and R₂₈ are independently selected from C₁₋₄alkyl, C₂₄alkenyl, hydroxy, —OC₁₋₄alkyl, halogen, cyano, nitro, —CF₃, —OCF₃, —SCH₃, —SO₂CH₃, —CO₂H, —CO₂CH₃, —C(═O)CH₃, phenyloxy and benzyloxy.

In compounds of formula (Ib), preferably

G₁ is —C(═O)—;

G₂ is CR₅R₆;

G₃ is —CR₇R₈;

R₅ is H or C₁₋₄alkyl;

R₆, R₇, and R₈ are hydrogen;

Ar is phenyl optionally substituted in the para position by R₁₈;

R₁₈ is selected from C₁₋₄alkyl, bromo, hydroxy, methoxy, A₁-NH-A₂-R₂₅, —O-A₁-NHC(═O)R₂₅, and —O—R₂₆;

R₂₅ is selected from hydrogen, C₁₋₆alkyl, phenyl, pyridyl, indolyl, napthyl and

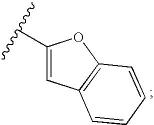

and

R₂₆ is selected from

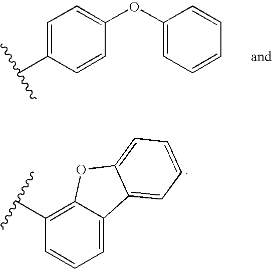

Even more preferred are compounds of formulas (I), (Ia) and (Ib) in which R₁ₐ and R_{1b} are both hydrogen.

Also, more preferred compounds are those of formulas (I), (Ia), and (Ib) in which A, B, and D are O.

In compounds of formula (I), or a pharmaceutically-acceptable salt, hydrate or prodrug thereof, preferably ring G is a spiroheterocyclo ring in which the ring G is selected from one of:

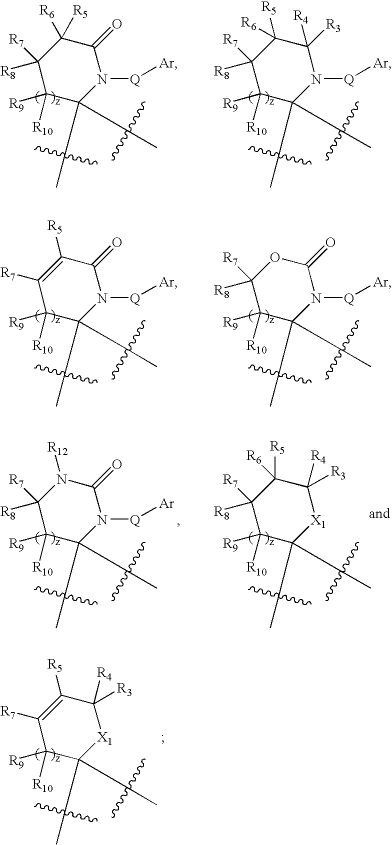

X₁ is selected from —S—, —S(═O)—, and —S(O)₂—;

Q is a bond or —CHR₁₄—, or C(═O);

Ar is aryl or heteroaryl optionally substituted with one to three R₁₈;

R₃, R₄, R₅, R₆, R₇, R₈, and R₉, are independently selected from the group consisting of hydrogen, halogen, nitro, cyano, amino, C₁₋₄alkyl, C₂₋₄alkenyl, hydroxy, C₁₋₄alkoxy, phenyloxy, benzyloxy, C₁₋₄alkylamino, C₁₋₄aminoalkyl, C₁₋₄hydroxyalkyl, C₃₋₇cycloalkyl, four to seven membered heterocyclo, five or six membered heteroaryl, phenyl, benzyl, phenyloxy, and benzyloxy;

R₁₂ is selected from hydrogen, or, C₁₋₄alkyl;

R₁₄ is hydrogen, halogen, C₁₋₄alkyl, OH, OCH₃, or NH₂;

r and s are independently selected from 0, 1, and 2; and

z is 0 or 1.

Methods of Preparation

Compounds of Formula I may be prepared by reference to the methods illustrated in the following Schemes 1 through 18. As shown therein, the end product is a compound having the same structural formula as Formula I. It will be understood that any compound of Formula I may be produced by Schemes 1 through 18 with suitable selection of appropriate substitutions. Solvents, temperatures, pressures, and other reaction conditions may readily be selected by one of ordinary skill in the art. In some cases the synthesis may be expedited by the use of protecting groups, as described, for example, in Greene and Wuts, Protective Groups in Organic Synthesis, (3rd Ed. 1999, John Wiley & Sons, New York, N.Y.). All documents cited are incorporated herein by reference in their entirety. Starting materials are commercially available or can be readily prepared by one of ordinary skill in the art.

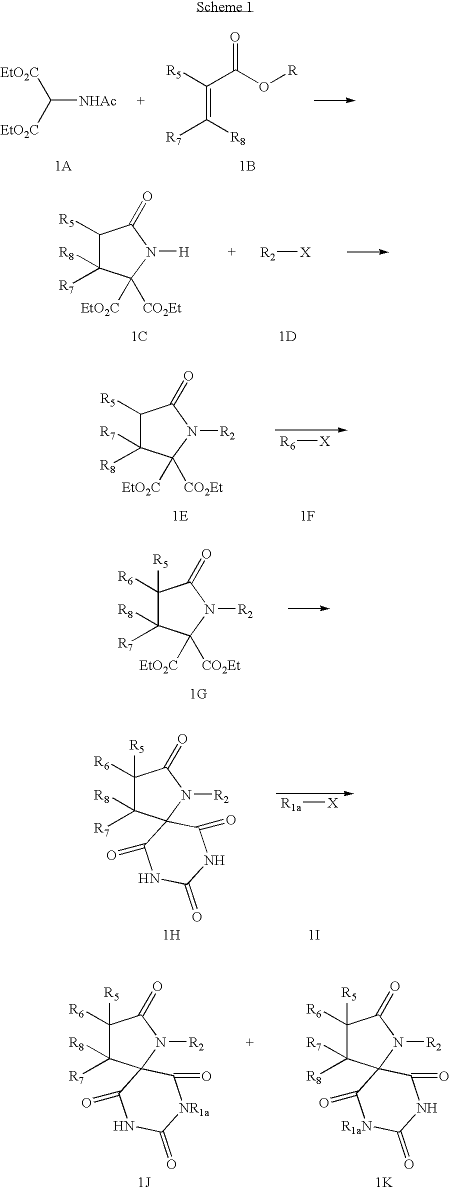

Compound 1C can be prepared by condensation of compounds 1A and 1B by known methods. See, e.g., Cocolas et al., J. Amer. Chem. Soc., Vol. 79 (1957), at p. 5203. Compound 1E can be prepared from compounds 1C and 1D (where X is a typical leaving group such as bromide, iodide and chloride), using standard alkylation conditions known in the art, such as NaH in DMF. Alternatively, compound 1E where R²is aryl group can be prepared from compounds 1C and 1D by using Pd(OAc)₂, diphenylphosphinoferrocene and NaOtBu (Shakespeare, Tetrahedron Lett., Vol. 30 [1999], at p. 2035). Compound 1G can be prepared from compounds 1E and 1F by using LiN(SiMe₃)₂ in THF. See, e.g., Ezquerra et al., Tetrahedron, Vol. 49 (1993), at p. 8665. Compound 1H can be prepared from compound 1G by reaction of urea and NaOEt in EtOH. See, e.g., Hildbrand et al., Helv. Chim. Acta, Vol. 79 (1996), at p. 702. Compounds 1J and 1K can be prepared from compounds 1H and an alkylating agent, 1I, by using bis(trimethylsilyl)acetamide and Bu₄NI in DCM. See, e.g., Benhida et al., Tetrahedron Lett., Vol. 37 (1996), at p. 1031.

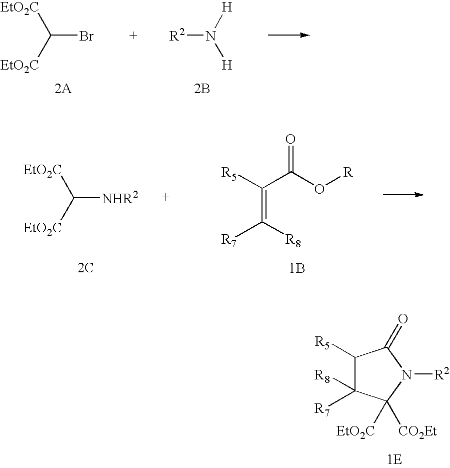

Alternatively to Scheme 1, compound 1E can be prepared as outlined in Scheme 2. Alkylation of an amine, 2B, with bromodiethylmalonate, 2A, produces compound 2C as reported in the literature (Balsiger et al., Helv. Chim. Acta, Vol. 36 (1953), at p. 708). Compound 1E can be prepared by condensation of compounds 2C and 1B in the presence of a base such as NaOEt in EtOH.

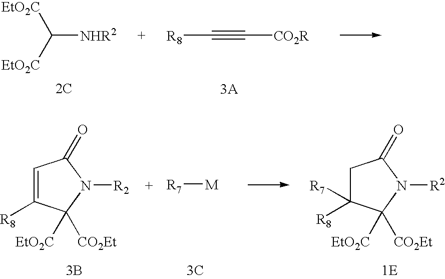

Alternatively, compound 1E can be prepared as shown in Scheme 3. Compound 3B can be prepared by condensation of 3A, with 2C in the presence of NaOEt and Bu4NBr in acetonitrile (Lopez et al., Tetrahedron, Vol. 52 [1996], at p. 8365). Compound 1E can be prepared from compound 3B by 1,4 addition of compound 3C, where M is a suitable metal-ligand complex that catalyzes the 1,4 addition (M═CuCN/MgBrR₄). See Berkowitz et al., J. Am. Chem. Soc., Vol. 118 (1996), at p. 9426. Alternatively many other nucleophiles are capable of successful Michael addition to compounds such as 3B, allowing for further diversification. Alkylation adjacent to the pyrrolidinone carbonyl will incorporate additional substituents as desired. See, e.g., Ezquerra et al., Tetrahedron, Vol. 49 (1993), at p. 8665.

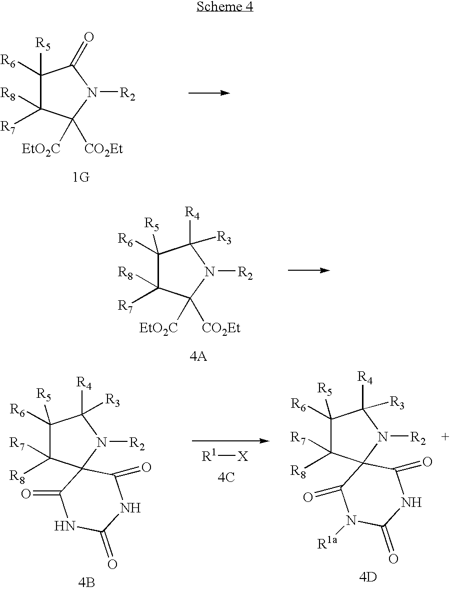

Compound 4A, where R₃═R₄═H, can be prepared from compound 1G by selective reduction of an amide by using BH₃ in THF. See, e.g., Jones et al., Bioorganic Med Chem Lett., Vol. 6(2) (1996), at p. 2399-2404. Compound 4B can be prepared from compound 4A by reaction of urea and NaOEt in EtOH as described above. Compounds 4D can be prepared as described in Scheme 1.

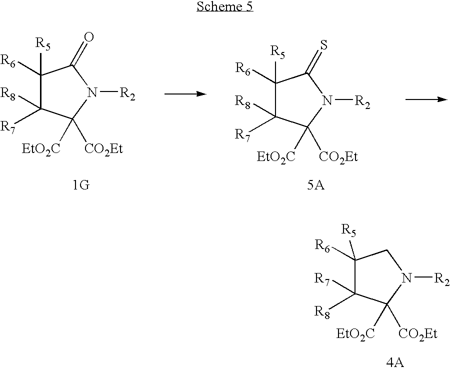

Alternatively, compound 4A can be prepared by reduction of 5A with Raney-Nickel. See, e.g., Kalaus et al., Liebigs Ann Chem., (1995), at pp. 1245-1251. Thioamide 5A can be prepared from compound 1G by reaction with Lawesson reagent. See, e.g., Sowinski et al., J. Org. Chem., Vol. 61 (1996), at p. 7671.

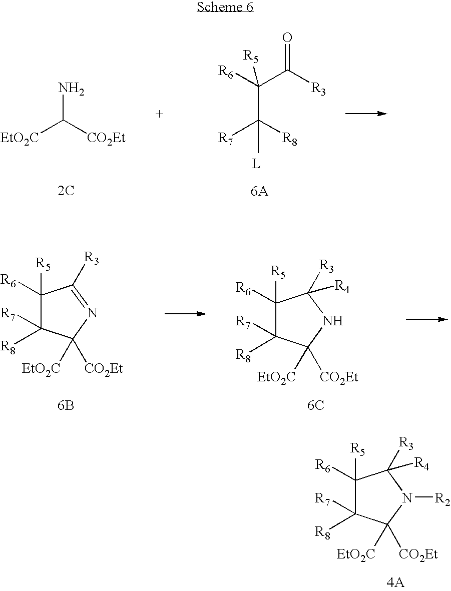

Alternatively, compound 4A can be prepared from compound 6C by alkylation with a suitable electrophile R²-L, (where L is a suitable leaving group such as a halide or sulfonate). Compound 6B can be prepared by condensation of 2C and 6B by heating in benzene with azeotropic removal of water. Compound 6C, where R₃ is hydrogen, can be prepared from compound 6B by reaction of NaBH₄ in acetonitrile/acetic acid (Cimarelli et al., J. Org. Chem., Vol. 61 (1996), at p. 5557). Compound 6C where R₃ is not hydrogen, can be prepared by reaction of BF₃—OEt₂ and R₃—MgCl (Imines, such as 6B, can react selectively with Grignard reagents in the presence of an ester functional group). See, e.g., Harwood et al., Synlett, Vol. 11 (1996), at p. 1051.

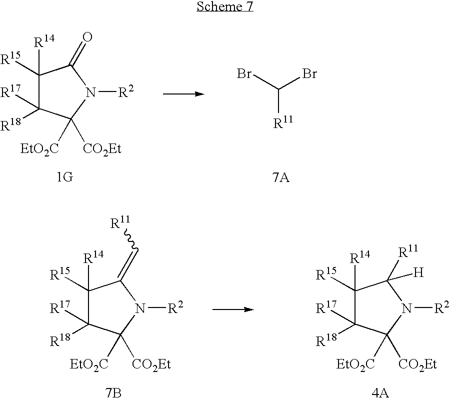

Alternatively, compound 4A can be prepared by reduction of enamine 7B with NaBH₄ in acetonitrile/acetic acid. See Cimarelli et al., J. Org. Chem., Vol. 61 (1996), at p. 5557. Reaction of 1G with 7A in the presence of Zn, TiCl₄ and TMEDA in THF will produce compound 7B. See, e.g., Takai et al., Tetrahedron Lett., Vol. 30 (1989), at p. 211.

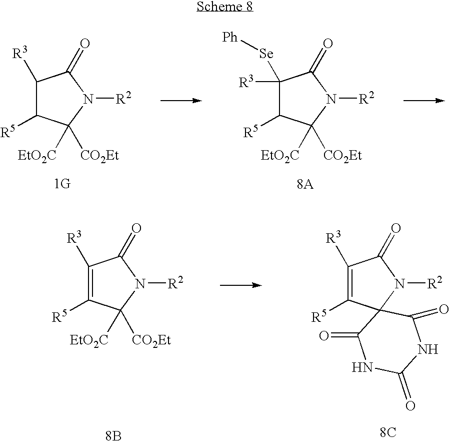

Reaction of 1G with LDA and PhSeCl will produce compound 8A. Treatment of 8A with H₂O₂ will produce 8B. See, e.g., Giovenzana et al., Tetrahedron Asymmetry, Vol. 8 (1997), at p. 515. Condensation of 8B with urea as described in Scheme 1 will provide 8C. Alternatively, compound 3B (see Scheme 3) can be condensed with urea to produce 8C.

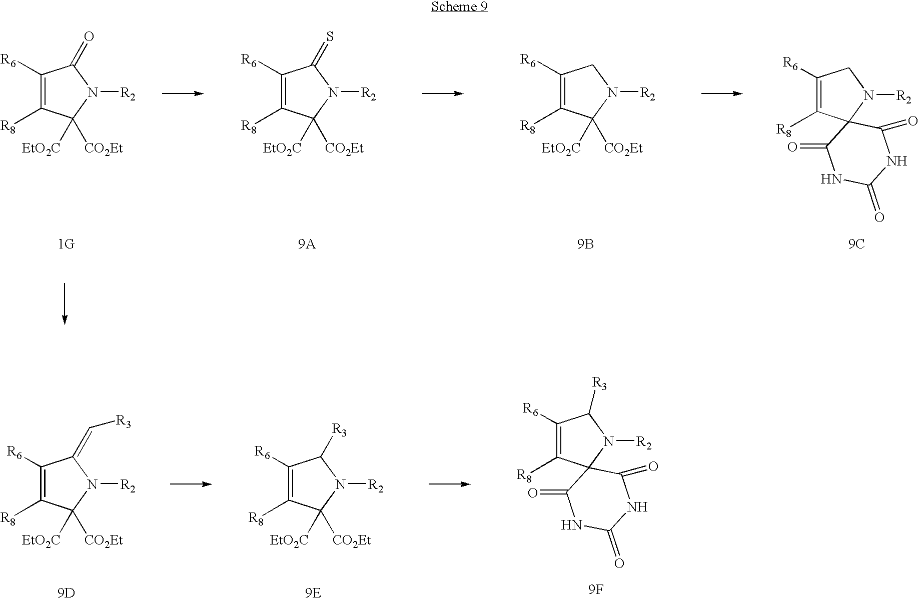

Reaction of compound 1G with Lawessons' reagent as described in Scheme 5, will provide thioamide 9A, which when reacted with Raney nickel will provide compound 9B. Condensation of 9B with urea as described in Scheme 1 will provide 9C. Alternatively, reaction of 1 G with a methylenedibromide reagent, in the presence of Zn and TiCl₄ as described for Scheme 7, will provide 9D, which can be reduced or condensed directly with urea to provide compound 9F.

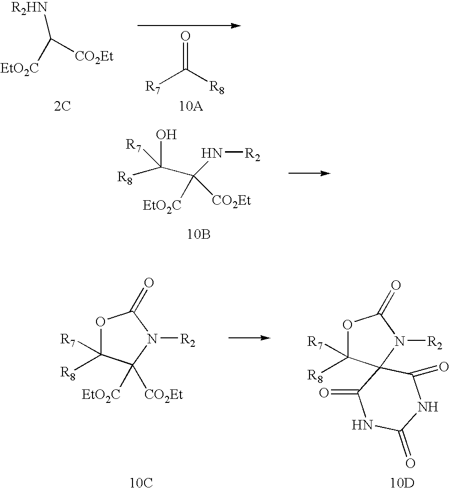

Aldol reaction of 2C and 10A (see, e.g., Walford et al., J. Med. Chem., Vol. 14 [1971], at p. 339) will produce 10B. Reaction of 10B with phosgene in THF (see, e.g., Gawley et al., J. Org. Chem., Vol. 61 [1996], at p. 8103), provides compound 10C. Condensation of 10C with urea as described in Scheme 1, provides compound 10D.

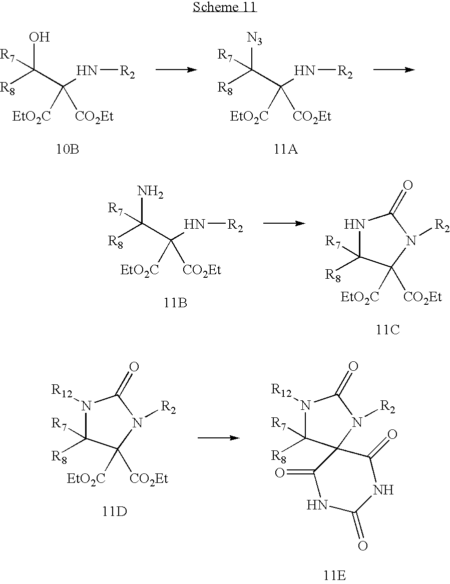

Reaction of compound 10B with CCl₄ and PPh₃ in THF (see, e.g., Ohba et al., Bioorg. Med. Chem. Lett., Vol. 6 [1996], at p. 219), will produce the chloride, or with tosyl chloride in the presence of pyridine, followed by reaction with NaN₃ in DMF, will produce compound 11A. Reduction of 11A either by Staudinger conditions, or by hydrogenation with a catalyst such as PtO₂ in EtOH (see, e.g., Lebarbier et al., Synthesis, Vol. 11 [1996], at p. 1371), will produce 11B. Reaction of 11B with phosgene, or heating with dimethylcarbonate, will provide 11C. Alkylation of the nitrogen in the presence of a suitable base such as LiHMDS will provide 11D. Condensation of 11C or 11D with urea as described in Scheme 1 will provide compound 11E.

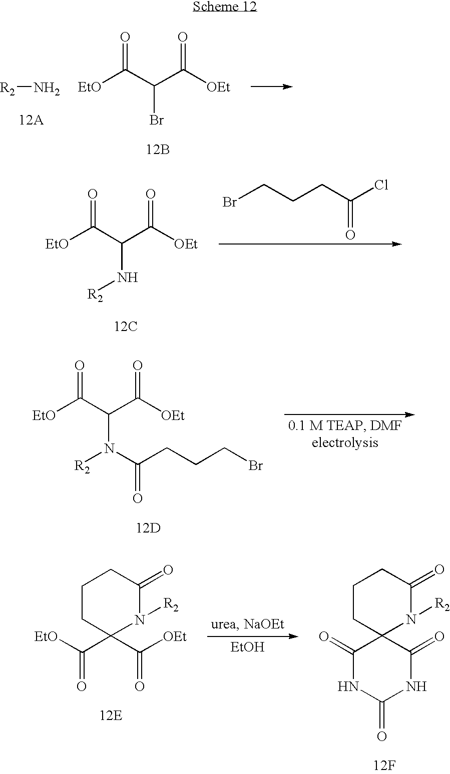

Amine 12A is alkylated with bromomalonate 12B, to produce intermediate 12C. Acylation with commercially available 4-bromobutyric acid chloride in the presence of triethylamine provides 12D. Intramolecular cyclization provides 12E (See, e.g., Casadei et al., Bull. Chim. Soc. Fr., Vol. 5 [1989], at pp. 650-656.) Condensation with urea as described in Scheme 1 provides 12F.

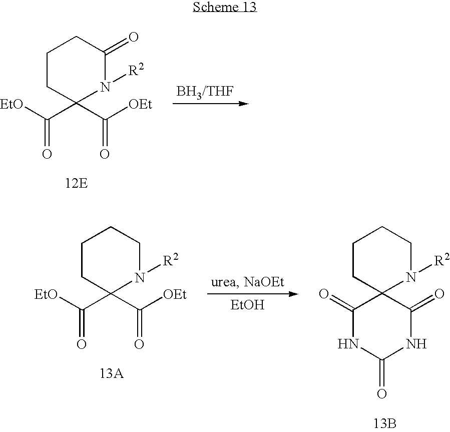

Intermediate 12E can be reduced with a variety of agents such as borane in THF, as described in Scheme 4, to produce compound 13A. Condensation of 13A with urea as described in Scheme 1 will provide compound 13B.

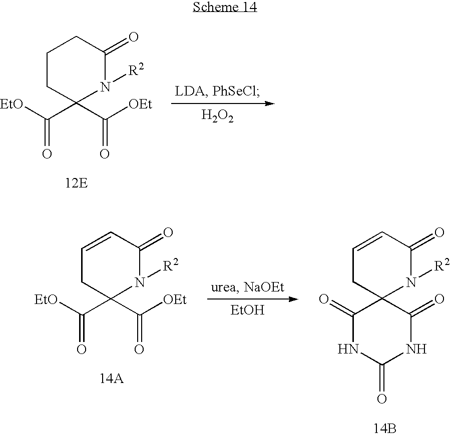

Intermediate 12E is deprotonated with LDA, followed by reaction with phenylselenium chloride. Oxidation with peroxide, followed by elimination provides intermediate compound 14A. Condensation with urea, as described in Scheme 1, provides compound 14B.

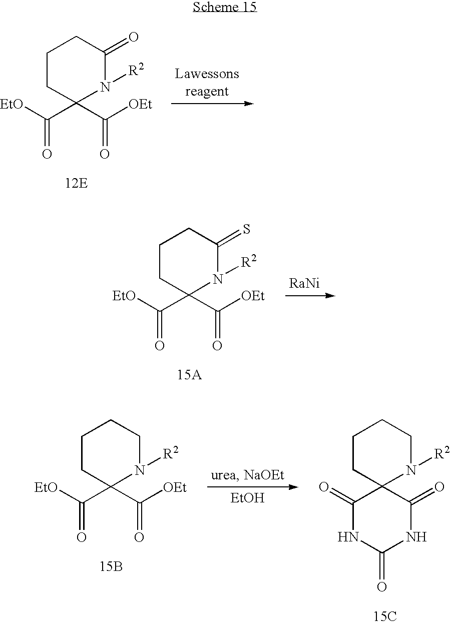

Intermediate 12E is reacted with Lawesson reagent to provide thioamide 15A. Reduction with Raney nickel provides intermediate 15B. Condensation with urea provides 15C.

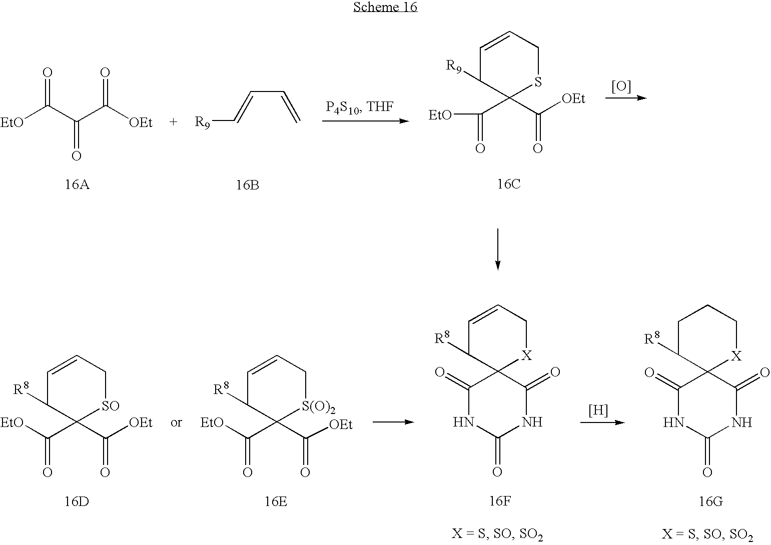

Reaction of Compound 16A with diene 16B in the presence of P₄S₁₀ provides intermediate 16C. See, e.g., Lawson et al., J. Chem. Soc. Perkin Trans. I, (1988), at pp 663-673. Oxidation of the sulfur atom by either mCPBA or oxone will provide either intermediate 16D or 16E. Condensation of intermediate 16C, 16D, or 16E, with urea will provide spirobarbiturate 16F. Reduction of the double bond can be effected by hydrogenation in the presence of a suitable catalyst such as palladium on carbon. In the case where the sulfur is unoxidized (—S—), reduction can be effected with Wilkinson's catalyst.

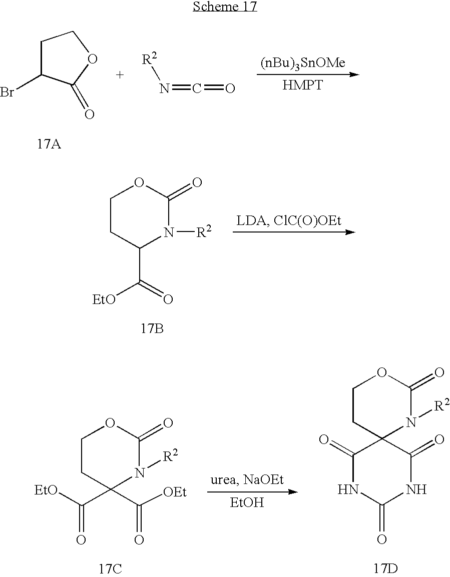

Reaction of intermediate 17A with an isocyanate will produce intermediate 17B (See, e.g., Shibata et al., “Synthesis of Six-Membered Heterocycles Via The Ring Clevage of a Bromo-butyrolactone Promoted by Organotin Alkoxide”, Synthesis, Vol. 6 (1988), at p. 486-488). Reaction of 17B with LDA followed by ethylchloroformate provides intermediate 17C. Condensation of 17C with urea provides spirobarbiturate 17D.

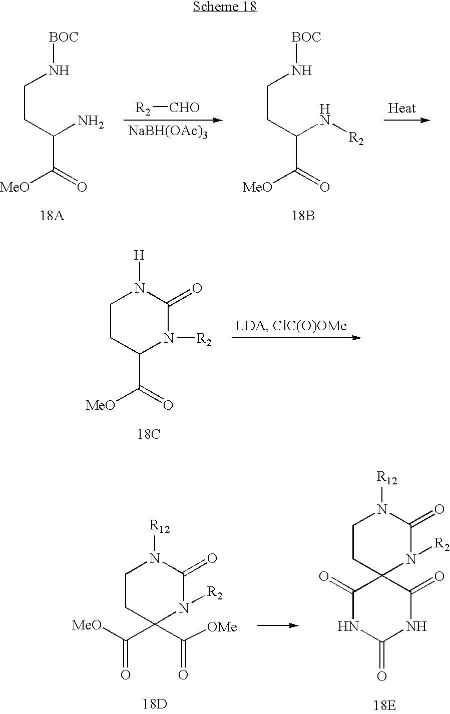

Commercially-available N-γ-Boc-L-diaminobutyric acid methyl ester reacted under reductive alkylation conditions provides intermediate 18B. Pyrolysis of intermediate 18B, or removal of the Boc group with trifluoroacetic acid followed by treatment with phosgene, provides intermediate 18D. Reaction with LDA and ethylchloroformate produces intermediate 18D. Condensation of 18D with urea provides spirobarbiturate 18E.

Utility

The compounds of formula I act as inhibitors of matrix metalloprotease (MMP) and/or TNF-α and/or aggrecanase activity. The MMP inhibitory activity of the compounds of the present invention has been demonstrated using assays of MMP activity, for example, using the assay described below for assaying inhibitors of MMP activity. The inhibition of aggrecanase, TNF-α, and other metalloproteases such as MMP-13 by molecules of the present invention should prevent the degradation of cartilage by these enzymes, thereby alleviating the pathological conditions of OA and RA. Thus, the compounds of formula I likely have the ability to suppress or inhibit cartilage degradation, for example, as demonstrated using the animal model of acute cartilage degradation described below. The term “treating” or “treatment” as used herein is intended to refer to responsive measures designed to alleviate or cure the disease or disorder and/or its symptoms, as well as prophylaxis or preventative measures designed to inhibit its development or the severity of the disorder or its symptoms.

The compounds of the present invention should have utility for treating and modifying the progression or prevention of osteoarthritis associated with matrix metalloprotease-mediated breakdown of cartilage and bone that occurs in patients with osteoarthritis.

The compounds of the present invention should also have utility for treating rheumatoid arthritis associated with matrix metalloprotease-mediated breakdown of cartilage and bone, and/or TNF-α associated progression of disease that occurs in patients with rheumatoid arthritis.

The compounds of the present invention should also have utility for treating osteopenia associated with matrix metalloprotease-mediated breakdown of cartilage and bone that occurs in osteoporosis patients.

Metalloproteases have also been implicated in the degradation of basement membranes to allow infiltration of cancer cells into the circulation and subsequent penetration into other tissues leading to tumor metastasis (Stetler-Stevenson, Cancer and Metastasis Reviews, 9, 289-303, 1990). The compounds of the present invention thus should be useful for treating invasive tumors by inhibition of metastasis.

Compounds that inhibit the production or action of TNF and/or aggrecanase and/or MMP's are potentially useful for treating various inflammatory, infectious, immunological, or malignant diseases or conditions. These include acute infection, acute phase response, Addison's disease (autoimmune disease of the adrenal glands); age related macular degeneration, allergy, aneurism, aortic aneurism, atherosclerosis, atopic dermatitis, autoimmune disease, autoimmune hepatitis, arthritis (such as rheumatoid arthritis, psoriatic arthritis or osteoarthritis), Bechet's disease, cachexia (including cachexia resulting from cancer or HIV), calcium pyrophosphate dihydrate deposition disease, cardiovascular effects, chronic fatigue syndrome, chronic obstruction pulmonary disease, coagulation, congestive heart failure, corneal ulceration, enteropathic arthropathy, Felty's syndrome, fever, fibromyalgia syndrome, fibrotic disease, gingivitis, glucocorticoid withdrawal syndrome, gout, graft versus host disease, including contact hypersensitivity, delayed-type hypersensitivity, and gluten-sensitive enteropathy (Celiac disease); contact dermatitis (including that due to poison ivy); Hashimoto's thyroiditis; Autoimmune Hyperthyroidism, such as Graves' Disease; hyperoxic alveolar injury, infectious arthritis, inflammation, inflammatory bowel disease, including ulcerative colitis and Crohn's disease; lupus (systemic lupus erythematosis); intermittent hydrarthrosis, HIV wasting syndrome, Lyme disease, meningitis, multiple sclerosis, myasthenia gravis, mycobacterial infection, neovascular glaucoma, osteoarthritis, pelvic inflammatory disease, periodontitis, polymyositis/dermatomyositis, post-ischaemic reperfusion injury, post-radiation asthenia, psoriasis, psoriatic arthritis, pydoderma gangrenosum, relapsing polychondritis, Reiter's syndrome, respiratory and pulmonary diseases including but not limited to asthma, exercise induced asthma, chronic obstructive pulmonary disease (COPD), emphysema, bronchitis, and acute respiratory distress syndrome (ARDS), rheumatic fever, rheumatoid arthritis (including juvenile rheumatoid arthritis and adult rheumatoid arthritis), sarcoidosis, scleroderma, sepsis syndrome, Sezary's syndrome, Still's disease, shock, Sjogren's syndrome, skin inflammatory diseases, solid tumor growth and tumor invasion by secondary metastases, spondylitis, stroke, systemic lupus erythematosus, ulcerative colitis, uveitis, vasculitis, autoimmune polyglandular disease (also known as autoimmune polyglandular syndrome); autoimmune alopecia; pernicious anemia; vitiligo; autoimmune hypopituatarism; Guillain-Barre syndrome; other autoimmune diseases; glomerulonephritis; serum sickness; uticaria; allergic diseases such as respiratory allergies (e.g., asthma, hayfever, allergic rhinitis) or skin allergies; scleracierma; mycosis fungoides; acute inflammatory and respiratory responses (such as acute respiratory distress syndrome and ishchemia/reperfusion injury); dermatomyositis; alopecia areata; chronic actinic dermatitis; eczema; Behcet's disease; Pustulosis palmoplanteris; Pyoderma gangrenum; atopic dermatitis; systemic schlerosis; and morphea and Wegener's granulomatosis.

Additionally, the inventive compounds may be useful as anti-cancer and/or anti-tumor agents. The compounds of the present invention are useful in treating tumor growth, as an adjunct to chemotherapy, and for treating cancer, more particularly, cancer of the lung, prostate, colon, breast, ovaries, and bone.

The compounds of the present invention may be administered alone or in combination with each other and/or other suitable therapeutic agents, and the methods of treating diseases associated with the production or action of TNF-α and/or aggrecanase and/or MMP's may comprise administering the compounds of Formula (I) alone or in combination with each other and/or other suitable therapeutic agents. The other therapeutic agents may comprise anti-tumor agents, immunosuppressants, antibodies, anti-inflammatory agents (steroidal and non-steroidal), TNF-α receptor inhibitors or anti-TNF-α antibodies, anti-cytokines, anti-IL-4 or IL-4 receptor fusion proteins, antioxidants, angiogenesis modulators, antiosteoporosis agents, hormone replacement therapies or hormone receptor modulators, oral contraceptives, antiproliferative agents, and/or inhibitors such as nuclear translocation inhibitors, p38 kinase inhibitors, phosphodiesterase inhibitors, and/or NF-kappa B inhibitors.

More particular examples of other suitable therapeutic agents to be used in combination with the inventive compounds include:

anti-tumor agents such as carmustine (BCNU), lomustine (CCNU), melphalan, buslfan, chlorambucil, doxorubicin, daunorubicin, idarubicin, cytarabine, fluorouracil, gemcitabine, capecitabine, Bleomycin, etoposide, teniposide, mitoxantrone, leucovoin, paclitaxel, docetaxel, vincristine, vinblastine, vinorelbine, mitomycin C, cisplatin, and carboplatin;

immunosuppressants such as cyclosporins (e.g., cyclosporin A), CTLA4-Ig, antibodies such as anti-ICAM-3, anti-IL-2 receptor (Anti-Tac), anti-CD45RB, anti-CD2, anti-CD3, anti-CD4, anti-CD80, anti-CD86, monoclonal antibody OKT3, agents blocking the interaction between CD40 and CD154, such as antibodies specific for CD40 and/or CD154 (i.e., CD40L), fusion proteins constructed from CD40 and CD154 (CD40Ig and CD8-CD154), interferon beta, interferon gamma, methotrexate, FK506 (tacrolimus, Prograf), rapamycin (sirolimus or Rapamune)mycophenolate mofetil, leflunomide (Arava), azathiprine and cyclophosphamide;

NF-kappa B inhibitors such as deoxyspergualin (DSG);

non-steroidal antiinflammatory drugs (NSAIDs) such as ibuprofen, cyclooxygenase-2 (COX-2) inhibitors such as celecoxib (Celebrex) and rofecoxib (Vioxx), or derivatives thereof;

steroids such as prednisone, dexamethasone, or gold compounds;

TNF-α inhibitors such as tenidap, anti-TNF antibodies or soluble TNF receptors such as etanercept (Enbrel);

inhibitors of p-38 kinase such as BIRB-796, RO-3201195, VX-850, and VX-750;

phosphodiesterase inhibitors, including PDE4 inhibitors such as Arofyline, Cilomilast, Roflumilast, C-11294A, CDC-801, BAY-19-8004, Cipamfylline, SCH351591, YM-976, PD-189659, Mesiopram, Pumafentrine, CDC-998, IC-485, and KW-4490, and PDE7 inhibitors such as IC242 (Lee et. al., “PDE7A is expressed in human B-lymphocytes and is up-regulated by elevation of intracellular cAMP, ” Cell Signalling, Vol. 14, [2002] at pp. 277-284) and compounds disclosed in WO 00/68230, WO 01/29049, WO 01/32618, WO 01/34601, WO 01/36425, WO 01/74786, WO 01/98274, and WO 02/28847;

anti-cytokines such as anti-IL-1 mAb or IL-1 receptor agonists, anti-IL-4 or IL-4 receptor fusion proteins; and

PTK inhibitors.

The above other therapeutic agents, when employed in combination with the compounds of the present invention, may be used, for example, in those amounts indicated in the Physicians' Desk Reference (PDR) or as otherwise determined by one of ordinary skill in the art. In the methods of the present invention, such other therapeutic agent(s) may be administered prior to, simultaneously with, or following the administration of the inventive compounds.

Compounds of the present invention have been shown to inhibit MMPs as determined by the assay described below.

Compounds of the present invention have been shown to inhibit TNF production in lipopolysacharride stimulated mice, for example, using the assay for TNF induction in mice and in PBMC's, and in human whole blood as described below.

Compounds of the present invention have been shown to inhibit aggrecanase, a key enzyme in cartilage breakdown, as determined by the aggrecanase assay described below.

Compounds of the present invention have been shown to inhibit the breakdown of cartilage in the articular cartilage explant assay described below.

Aggrecanase Enzymatic Assay

A novel enzymatic assay was developed to detect potential inhibitors of aggrecanase. The assay uses active aggrecanase accumulated in media from stimulated bovine nasal cartilage (BNC) or related cartilage sources and purified cartilage aggrecan monomer or a fragment thereof as a substrate.

The substrate concentration, amount of aggrecanases time of incubation and amount of product loaded for Western analysis were optimized for use of this assay in screening putative aggrecanase inhibitors. Aggrecanase is generated by stimulation of cartilage slices with interleukin-1 (L-1), tumor necrosis factor alpha (TNF-α) or other stimuli. Matrix metalloproteases (MMPs) are secreted from cartilage in an inactive, zymogen form following stimulation, although active enzymes are present within the matrix. We have shown that following depletion of the extracellular aggrecan matrix, active MMPs are released into the culture media (Tortorella et al., Trans. Ortho. Res. Soc., Vol. 20 (1995), at pp. 341). Therefore, in order to accumulate BNC aggrecanase in culture media, cartilage is first depleted of endogenous aggrecan by stimulation with 500 ng/ml human recombinant IL-β for 6 days with media changes every 2 days. Cartilage is then stimulated for an additional 8 days without media change to allow accumulation of soluble, active aggrecanase in the culture media. In order to decrease the amount of other matrix metalloproteases released into the media during aggrecanase accumulation, agents which inhibit MMP-1, -2, -3, and -9 biosynthesis are included during stimulation. This BNC conditioned media, containing aggrecanase activity is then used as the source of aggrecanase for the assay. Aggrecanase enzymatic activity is detected by monitoring production of aggrecan fragments produced exclusively by cleavage at the Glu373-Ala374 bond within the aggrecan core protein by Western analysis using the monoclonal antibody, BC-3 (Hughes et al., Biochem. J., Vol. 306 (1995), at 799-804). This antibody recognizes aggrecan fragments with the N-terminus, 374ARGSVIL, generated upon cleavage by aggrecanase. The BC-3 antibody recognizes this neoepitope only when it is at the N-terminus and not when it is present internally within aggrecan fragments or within the aggrecan protein core. Other proteases produced by cartilage in response to IL-1 do not cleave aggrecan at the Glu373-Ala374 aggrecanase site; therefore, only products produced upon cleavage by aggrecanase are detected. Kinetic studies using this assay yield a Km of 1.5±0.35 uM for aggrecanase.

To evaluate inhibition of aggrecanase, compounds are prepared as 10 mM stocks in DMSO, water or other solvents and diluted to appropriate concentrations in water. Drug (50 ul) is added to 50 ul of aggrecanase-containing media and 50 ul of 2 mg/ml aggrecan substrate and brought to a final volume of 200 ul in 0.2 M Tris, pH 7.6, containing 0.4 M NaCl and 40 mM CaCl₂. The assay is run for 4 hr at 37° C., quenched with 20 mM EDTA and analyzed for aggrecanase-generated products. A sample containing enzyme and substrate without drug is included as a positive control and enzyme incubated in the absence of substrate serves as a measure of background.

Removal of the glycosaminoglycan side chains from aggrecan is necessary for the BC-3 antibody to recognize the ARGSVIL epitope on the core protein. Therefore, for analysis of aggrecan fragments generated by cleavage at the Glu373-Ala374 site, proteoglycans and proteoglycan fragments are enzymatically deglycosylated with chondroitinase ABC (0.1 units/10 ug GAG) for 2 hr at 37° C. and then with keratanase (0.1 units/10 ug GAG) and keratanase II (0.002 units/10 ug GAG) for 2 hr at 37° C. in buffer containing 50 mM sodium acetate, 0.1 M Tris/HCl, pH 6.5. After digestion, aggrecan in the samples is precipitated with 5 volumes of acetone and resuspended in 30 ul of Tris glycine SDS sample buffer (Novex) containing 2.5% beta mercaptoethanol. Samples are loaded and then separated by SDS-PAGE under reducing conditions with 4-12% gradient gels, transferred to nitrocellulose and immunolocated with 1:500 dilution of antibody BC3. Subsequently, membranes are incubated with a 1:5000 dilution of goat anti-mouse IgG alkaline phosphatase second antibody and aggrecan catabolites visualized by incubation with appropriate substrate for 10-30 minutes to achieve optimal color development. Blots are quantitated by scanning densitometry and inhibition of aggrecanase determined by comparing the amount of product produced in the presence versus absence of compound.

MMP Assays

The enzymatic activities of recombinant MMP-1, 2, 3, 7, 8, 9, 13, 14, 15, and 16 were measured at 25° C. with a fluorometric assay (Copeland et al., Bioorganic Med. Chem. Lett. Vol. 5 (1995), at 1947-1952). Final enzyme concentrations in the assay were between 0.05 and 10 nM depending on the enzyme and the potency of the inhibitor tested. The permisive peptide substrate, MCA-Pro-Leu-Gly-Leu-DPA-Ala-Arg-NH₂, was present at a final concentration of 10 μM in all assays. Initial velocities, in the presence or absence of inhibitor, were measured as slopes of the linear portion of the product progress curves. IC₅₀ values were determined by plotting the inhibitor concentration dependence of the fractional velocity for each enzyme, and fitting the data by non-linear least squares methods to the standard isotherm equation (Copeland, Enzymes: A Practical Introduction to Structure, Mechanism and Data Analysis [Wiley-VHC, N.Y., 1996], at pp 187-223). All of the compounds studied here were assumed to act as competitive inhibitors of the enzyme, and based on the assumption of competitive inhibition, the IC₅₀ values were converted to Kᵢ values as previously described.

Compounds tested in the above assay are considered to be active if they exhibit a Kᵢ of ≦10 μM. Preferred compounds of the present invention have Kᵢ's of ≦1 μM. More preferred compounds of the present invention have Kᵢ's of ≦0.1 μM. Even more preferred compounds of the present invention have Kᵢ's of ≦0.01 μM.

TNFα Secretion Assay

The ability of compounds to inhibit the production and secretion of TNFα from leukocytes was performed using either PBMC (obtained as described above) or the THP-1 cell line as a source of monocytes. Compounds were diluted in RPMI 1640 supplemented with 10% FBS and DMSO at a final concentration of 0.2%. Cells (2×10⁵/well in U-bottom 96 well plates) were pre-incubated with compounds for 30 min at 37° C. prior to addition of lipopolysaccharide (LPS) at a final concentration of 6.25 ng/ml in a total volume of 200 μL. After 4 h at 37C., 50 μL of supernatant was carefully aspirated for detection of soluble TNF-α. Soluble TNF-α was detected by ELISA developed by R&D Systems (Minneapolis, Minn.) according to the manufacturers instructions.

TNF Human Whole Blood Assay

Blood is drawn from normal donors into tubes containing 143 USP units of heparin/10 ml. 225 ul of blood is plated directly into sterile polypropylene tubes. Compounds are diluted in DMSO/serum free media and added to the blood samples so the final concentration of compounds are 50, 10, 5, 1, 0.5, 0.1, and 0.01 μM. The final concentration of DMSO does not exceed 0.5%. Compounds are preincubated for 15 minutes before the addition of 100 ng/ml LPS. Plates are incubated for 5 hours in an atmosphere of 5% CO₂ in air. At the end of 5 hours, 750 ul of serum free media is added to each tube and the samples are spun at 1200 RPM for 10 minutes. The supernatant is collected off the top and assayed for TNF-α production by a standard sandwich ELISA. The ability of compounds to inhibit TNF-α production by 50% compared to DMSO treated cultures is given by the IC₅₀ value.

TNF Induction in Mice

Test compounds are administered to mice either I.P. or P.O. at time zero. Immediately following compound administration, mice receive an I.P. injection of 20 mg of D-galactosamine plus 10 μg of lipopolysaccharide. One hour later, animals are anesthetized and bled by cardiac puncture. Blood plasma is evaluated for TNF levels by an ELISA specific for mouse TNF. Administration of representative compounds of the present invention to mice results in a dose-dependent suppression of plasma TNF levels at one hour in the above assay.

Bovine Articular Cartilage Explant Assay.

This assay is performed as described below with some modifications of the procedure of Badger et al., Osteoarthritis and Cartilage, Vol. 8 (2000), at pp. 434-443. Intact carpal joints from calves (1-3 months old) are dissected exposing the cartilage. Cartilage disks are obtained by first scoring the cartilage surface with a 4 mm biopsy punch then dissecting away from the bone with a scalpel. Disks are washed with PBS containing 5× antibiotic/antimycotic solution and 250 ug/mL gentamyacin. Disks are placed in a petri dish along with DMEM, 10% FBS, 3× antibiotic/antimycotic solution and 150 ug/mL gentamyacin and incubated at 37° C. overnight. Four disks weighing a total of approximately 75 mg are placed into each well of a 24 well tissue culture plate containing 1 mL media (DMEM, 0.5% FBS, 3× antibiotic/antimycotic solution, 150 ug/mL gentamyacin). Disks are incubated at 37° C. for 72 hrs. Media is removed and replaced with 1 mL fresh media containing test compounds, 20 ng/mL rhIL-1α, and appropriate controls. Culture supernatants are removed every 3-4 days and replaced with media containing fresh compounds and rhEL-1α. Proteoglycan fragments in the culture supernatants are quantitated using dimethylmethylene blue which binds to and produces a color reaction with the sulfate groups of the proteoglycan fragments. Farndale et al. Biochem. Biophys. Acta., Vol. 883 (1986), at pp. 173-177. Collagen fragments in the supernatants are quantitated with Ehrlich's reagent which produces a color reaction with oxidized hydroxyproline, the unique amino acid found in collagen. Bergman et al., Anal. Biochem., Vol. 35 (1963), at pp. 1961-1965.

### EXAMPLES

The following examples illustrate preferred embodiments of the present invention and do not limit the scope of the present invention which is defined in the claims. Abbreviations employed herein are defined below.

| Abbreviations | |
|---|---|
| Ac | Acetyl |
| AcOH | Acetic acid |
| aq. | Aqueous |
| CDI | Carbonyldiimidazole |
| Bn | Benzyl |
| Bu | Butyl |
| Boc or BOC | tert-butoxycarbonyl |
| BOP reagent | Benzotriazol-1-yloxy- |
| | tris(dimethylamino) |
| | phosphonium |
| | hexafluorophosphate |
| DCM | dichloromethane |
| DIEA or Hunigs Base | Diisopropyl ethyl amine |
| DMAP | Dimethylaminopyridine |
| DMA | N,N-Dimethylacetamide |
| DMF | dimethylformamide |
| DMSO | Dimethylsulfoxide |
| EDC | 1-(3-Dimethylaminopropyl)- |
| | 3-ethylcarbodiimide |
| | hydrochloride |
| EtOAc | Ethyl acetate |
| Et | Ethyl |
| EtOH | Ethanol |
| H | Hydrogen |
| h | Hours |
| i | iso |
| HPLC | High pressure |
| | liquid chromatography |
| HOAc | Acetic acid |
| Lawesson's Reagent | [2,4-bis(4-methoxyphenyl)- |
| | 1,3-dithia-2,4-diphosphetane- |
| | 2-4-disufide |
| LC | liquid chromatography |
| Me | Methyl |
| MeOH | Methanol |
| min. | Minutes |
| M⁺ | (M + H)⁺ |
| M⁻¹ | (M + H)⁺ |
| MS | Mass spectrometry |
| Pd/C | Palladium on carbon |
| Ph | Phenyl |
| Pr | Propyl |
| Ret t. | Retention time |
| rt or RT | Room temperature |
| sat. | Saturated |
| S-Tol-BINAP | (S)-(−)-2,2′-Bis |
| | (di-p-tolylphosphino)- |
| | 1,1′-binapthyl |
| t | tert |
| TFA | Trifluoroacetic acid |
| THF | Tetrahydrofuran |
| YMC | YMC Inc, Wilmington, NC 28403 |

### Example 1

1-(4-Bromophenyl)-1,7,9-triazaspiro[4,5]decane-2,6,8,10-tetrone

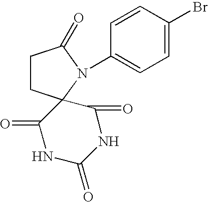

Step A: 2-[(4-Bromophenyl)amino]malonate diethyl ester

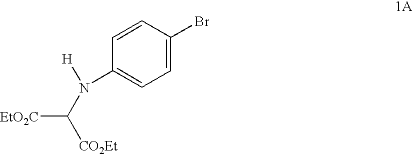

4-Bromoaniline (12.4 mmol, 2.14 g) and diethylbromomalonate (12.4 mmol, 2.1 ml) in DMF (25 ml) was heated at 100° C. for 16 hours. The reaction mixture was cooled, concentrated and purified by flash chromatography (SiO₂, 20% EtOAc/hexanes) to isolate compound 1A as a white solid (1.7 g, 41%). MS (ESI): 330 (M+H).

Step B: N-(4-Bromophenyl)-5,5-diethoxycarbonylpyrrolidone

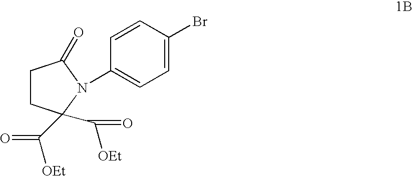

Compound 1A (1.38 g, 4.18 mmol) and NaOEt (24 mg, freshly prepared as a solution in EtOH) in EtOH (20 ml) was treated with ethyl acrylate (680 μl, 6.27 mmol). The reaction mixture was heated at 80° C. for 3 hours, then concentrated and purified by flash chromatography (SiO₂, 50% EtOAc/hexanes) to isolate compound 1B as a clear film (1.13, 71%).

Step C

Urea (1.0 mmol, 60 mg) was added to sodium (18.3 mg, 0.40 mmol) dissolved in EtOH (5 ml). After the reaction mixture was stirred at RT for 30 minutes, compound 1B (153 mg, 0.40 mmol) was added and the mixture heated at 80° C. for 5 hours. The reaction mixture was concentrated, dissolved in water (10 ml), acidified to pH 2 with 1N HCl and extracted with EtOAc (3×10 ml). The organic layer was dried over Na₂SO₄, concentrated, and purified by reverse phase HPLC to isolate Example 1 as a white solid (67 mg, 48%). MS (ESI): 352 (M−H).

### Example 2

1-Phenyl-1,7,9-triazaspiro[4,5]decane-2,6,8,10-tetrone

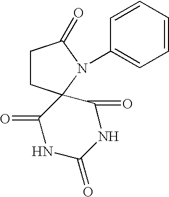

Palladium (5 mg, 10% in activated carbon) was added to Example 1 (8.3 mg, 0.024 mmol) dissolved in EtOH (1 ml). The reaction mixture was stirred under an atmosphere of hydrogen gas (1 atm) for 1 hour, then filtered through Celite, concentrated and purified by flash chromatography (SiO₂, 10% MeOH/EtOAc/1% AcOH) to provide Example 2 as a white solid (0.7 mg, 11%). MS (ESI): 272 (M−H).

### Example 3

1-[4-(1,4-biphenyl)phenyl]-1,7,9-triazaspiro[4,5]decane-2,6,8,10-tetrone

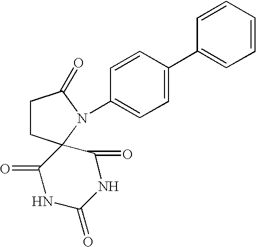

Step A: N-(4-(phenyl)phenyl)-5,5-diethoxycarbonylpyrrolidone

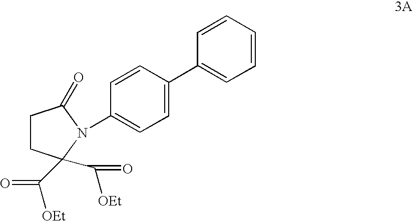

1,1′-Bis(diphenylphosphino)ferrocene]dichloropalladium(II)

DCM (30 mg, 0.035 mmol) was added to a solution of compound 2B (134 mg, 0.35 mmol), phenylboronic acid (85 mg, 0.7 mmol), and potassium phosphate (223 mg, 1.05 mmol) in DMF (1 ml). The reaction mixture was heated at 65° C. for 4 hours, then concentrated and purified by flash chromatography (SiO₂, 50% EtOAc/hexanes) to provide compound 3A as a clear film (115 mg, 86%).

Step B:

Compound 3A (131 mg, 0.34 mmol) was cyclized using the same or similar procedure described in Example 1, Step C, to isolate Example 3 as a white solid (7.2 mg, 6%). MS (ESI): 348 (M−H).

### Example 4

1-(4-Hydroxyphenyl)-1,7,9-triazaspiro[4,5]decane-2,6,8,10-tetrone

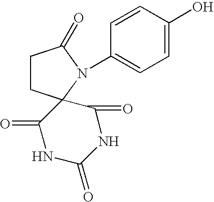

Step A: 1 2-[(4-(Benzyloxy)phenyl)amino]malonate diethyl ester

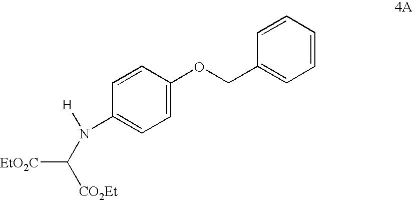

4-Benzyloxyaniline.HCl (86.1 mmol, 20.3 g), diethylbromomalonate (86.1 mmol, 14.7 ml) and N,N-diisopropylethylamine (86.1 mmol, 15 ml) in toluene (200 ml) was heated at 100° C. for 36 hours. The reaction mixture was concentrated, dissolved in water (500 ml) and extracted with EtOAc (3×300 ml). The organic phase was dried over Na₂SO₄, concentrated and purified by flash chromatography (SiO₂, 50% EtOAc/hexanes) to provide compound 4A as a brown solid (13.7 g, 45%).

Step B: N-(4-(Benzyloxy)phenyl)-5,5-diethoxycarbonylpyrrolidone

The same or similar procedure as in Example 1, Step B was followed to prepare the intermediate N-(4-(Benzyloxy)phenyl)-5,5-diethoxycarbonylpyrrolidone (6.63 g, 87%) from compound 4A (6.64 g, 18.6 mmol), except after heating, the mixture was concentrated, dissolved in water (500 ml,) extracted with DCM (3×250 ml), and the organic phase dried over Na₂SO₄, before concentration and purification by flash chromatography.

Step: 1-(4-Benzyloxyphenyl)-1,7,9-triazaspiro[4,5]decane-2,6,8,10-tetrone

The diethoxycarbonyl intermediate (4B) was then cyclized according to the same or similar procedure as described in Example 1. to provide Example 4C as a white solid (57 mg, 46%). MS (ESI): 378 (M−H).

Step D:

Palladium (5 mg, 10% on carbon) was added to Example 5 (20 mg, 0.053 mmol) and ammonium formate (55 mg, 1.2 mmol) in DMF (1 ml). The reaction mixture was heated at 40° C. for 20 minutes and filtered through a pad of silica gel. The filtrate was concentrated, diluted in EtOAc (50 ml) and washed with water (20 ml). The organic phase was dried over Na₂SO₄ and concentrated to give Example 4 as a white solid (4.8 mg, 32%). MS (ESI): 288 (M−H).

### Example 5

1-(4-Methoxyphenyl)-1,7,9-triazaspiro[4,5]decane-2,6,8,10-tetrone

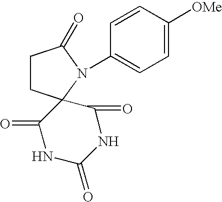

Step A: N-(4-(Hydroxy)phenyl)-5,5-diethoxycarbonylpyrrolidone

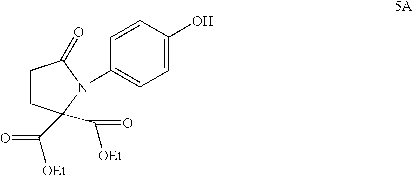

Palladium (91 mg, 10% on carbon) was added to compound 4B (1000 mg, 2,4 mmol) in DMF (20 ml) under 1 atm of hydrogen gas. The reaction mixture was stirred at RT for 1 hour, filtered through a pad of silica gel, concentrated and purified by flash chromatography (SiO₂, 65% EtOAc/hexanes) to isolate compound 5A as a white solid (718 mg, 92%). MS (ESI): 322 (M+H).

Step B: N-(4-(Methoxy)phenyl)-5,5-diethoxycarbonylpyrrolidone

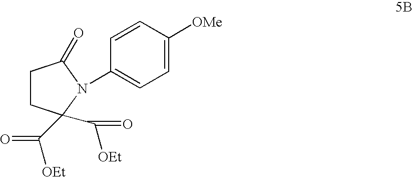

K₂CO₃ (16 mg, 0.12 mmol) was added to compound 5A (38 mg, 0.12 mmol) and methyl iodide (17 mg, 0.12 mmol) in acetone (1.2 ml). The reaction was heated at 40° C. for 18 hours, then diluted in EtOAc (50 ml), washed with sat. NaHCO₃ (30 ml), dried over Na₂SO₄, concentrated and purified by reverse phase HPLC to isolate compound 5B as a clear oil (23 mg, 58%).

Step C:

Urea (0.17 mmol, 10 mg) was added to sodium (3.2 mg, 0.14 mmol) dissolved in EtOH (0.25 ml). The reaction mixture was stirred at RT for 60 minutes, compound 5B (23 mg, 0.07 mmol) was added, and then the mixture was heated at 80° C. for 5 hours, diluted with 1N HCl (0.14 ml), and purified by reverse phase HPLC to isolate Example 5 as a white solid (20 mg, 95%). MS (ESI): 302 (M−H).

### Example 6

1-[4-(4-(methylthio)-1-phenoxy)phenyl]-1,7,9-triazaspiro[4,5]decane-2,6,8,10-tetrone

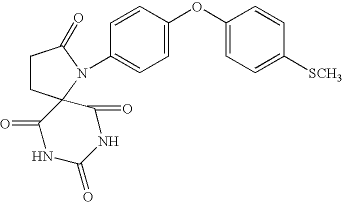

Step A:

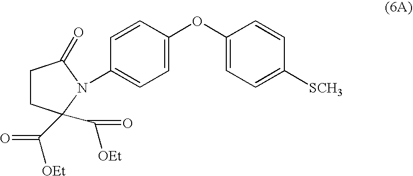

TEA (0.11 ml, 0.78 mmol) was added to compound 5A (50 mg, 0.16 mmol), 4-(methylthio)phenyl boronic acid (31 mg, 0.187 mmol), Cu(AcO)₂ (34 mg, 0.19 mmol), and 4A molecular sieves (50 mg) in DCE (1.25 ml). After 48 hours at rt, an additional 4-(methylthio)phenyl boronic acid (31 mg, 0.187 mmol) was added. The reaction mixture was stirred for 3 hours, filtered through silica gel, washed with EtOAc (1.0 ml), and concentrated.

Step B:

A sodium ethoxide/urea solution (0.71 M) was prepared by first dissolving sodium (163 mg, 7.1 mmol) in absolute EtOH (10 ml), followed by the addition of urea (533 mg, 8.88 mmol). The urea/sodium ethoxide solution (0.7 ml, 0.47 mmol of urea) was added to compound 6A. The reaction mixture was heated at 78° C. for 16 hours, then quenched with 1N HCl (0.47 ml) and purified stepwise by reverse phase prep HPLC and flash chromatography (SiO₂, 70% EtOAc/Hexanes/1% AcOH) to provide Example 6 as a film (11.6 mg, 17%, 2 steps).

### Example 7

3-(R,S)-Methyl-1-(1-phenoxyphenyl)-1,7,9-triazaspiro[4,5]decane-2,6,8,10-tetrone

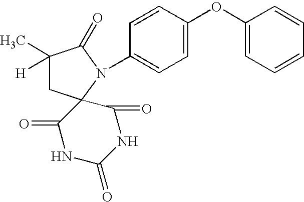

The procedure of Example 1 was followed, except ethyl methacrylate was used in place of ethyl acrylate (18 mg, 41%) to isolate Example 7 as a clear oil, MS (ESI): 378 (M−H).

### Example 8

4-(R)-Ethyl-1-(1-phenoxyphenyl)-1,7,9-triazaspiro[4,5]decane-2,6,8,10-tetrone and 4-(S)-Ethyl-1-(1-phenoxyphenyl)-1,7,9-triazaspiro[4,5]decane-2,6,8,10-tetrone

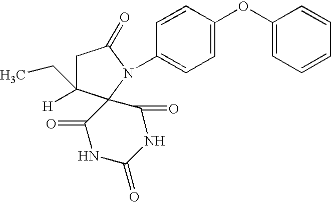

Example 8 was prepared in a similar manner to Example 1 with the exception that methyl-2-pentenoate was used in place of ethyl acrylate. The racemic product was then separated into the corresponding enantiomers using chiral reverse phase HPLC; isomer 1 [MS (ESI): 392 (M−H)], and isomer 2 [MS (ESI): 392 (M−H)].

### Example 9

1-(4-(((((Benzofuran-2-yl)carbonyl)amino)ethyl)oxy)phenyl)-1,7,9-triazaspiro[4,5]decane-2,6,8,10-tetrone

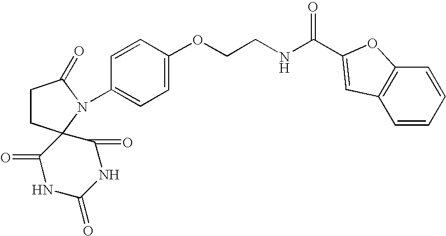

Step A:

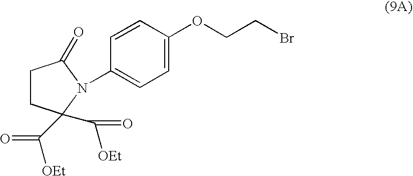

Compound 5A (727 mg, 2.262 mmol) was dissolved in 5.0 mL of dibromoethane and potassium carbonate (3.393 mmol, 469 mg) was added. The mixture was heated at reflux for 10 h, allowed to cool to RT, and the solvent removed under vacuum. Silica gel chromatography of the crude product (eluent 50% EtOAc/hex) and removal of the eluent under vacuum afforded 780 mg (81%) of compound 9A as a clear oil. (M+H=427.9).

Step B:

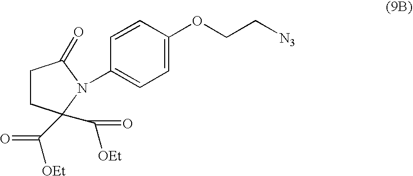

Compound 9A (281 mg, 0.656 mmol) was dissolved in 5.0 mL DMF. Sodium azide (1.312 mmol, 85 mg) was added, and then stirring of the mixture overnight at RT (18 h), resulted in complete reaction of the starting material. The solvent was removed under vacuum and silica gel chromatography of the crude product (eluent 50% EtOAc/hex) provided 207 mg (81%) of compound 9B as a cloudy film. (M+H=391.1).

Step C:

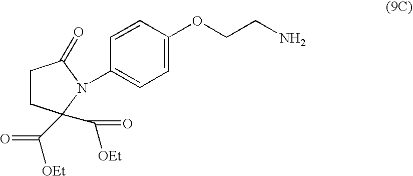

Compound 9B (118 mg, 0.302 mmol) was dissolved in a mixture of 4.0 mL THF and 1.0 mL water and then trimethylphosphine solution 1.0M in THF (0.039 ml, 0.39 mmol) was added. The reaction mixture was stirred at RT and after 40 minutes, an additional 0.39 mL trimethylphosphine solution was added and the reaction mixture stirred at RT for 2 h. The solvents were removed under vacuum, the crude product chromatographed using silica gel (eluent 1% TEA, 8% MeOH in chloroform), and the solvents removed under vacuum to afford compound 9C (47 mg, 43%) as a white crystalline solid. (M+H=365.22)

Step D:

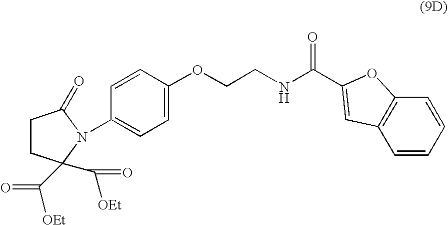

To compound 9C (47 mg, 0.129 mmol) dissolved in DMF (1.5 ML) was added 2-Benzofuran carboxylic acid (21 mg, 0.129 mmol), BOP reagent (57 mg, 0.129 mmol) and DIEA (34 μL, 25 mg, 0.193 mmol). The reaction mixture was stirred at RT for 1 h, the solvent removed under vacuum, and the crude product purified by column chromatography using silica gel (eluent 25% EtOAc:hexanes). The solvent was removed under vacuum to afford 9D (56 mg, 85%), as a clear film (M+H=509.15).

Step E:

Compound 9D (56 mg, 0.110 mmol) and the sodium salt of urea (0.96M) in EtOH (0.46 mL, 0.440 mmol) were sealed in a vial with a TEFLON® cap and heated in a oil bath at 80° C., with shaking for 24 h. The reaction mixture was allowed to cool to RT, and 1N HCl (0.45 mL) was added. The product was purified on prep HPLC to afford Example 9 (22 mg, 42%) as an off-white powder (M−H=475.0).

### Example 10

1-[4-(((4-Phenyloxy)phenyl)amino)phenyl]-1,7,9-triazaspiro[4.5]decane-2,6,8,10-tetrone

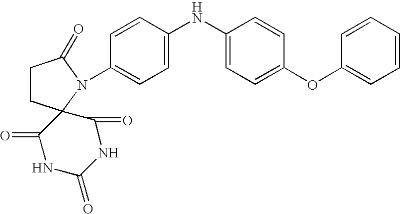

Step A:

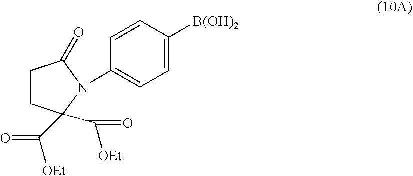

Compound 1B (100 mg, 0.26 mmol), bis(pinacolato)diboron (72 mg, 0.29 mmol) and tetrakistriphenylphospine palladium (0) (5 mg, 0.004 mmol) were heated in toluene at reflux for 2 hrs. The reaction mixture was washed with 1N HCl, the organic layer dried over magnesium sulfate and evaporated, and the residue purified by reverse phase HPLC to provide compound 10A (67 mg, 73%) as a colorless oil.

Step B:

Compound 10A (67 mg, 0.192 mmol), 4-Phenoxyaniline (36 mg, 0.192 mmol), copper acetate (35 mg, 0.192 mg), ˜40 mg 4A crushed molecular sieves, and TEA (0.13 mL, 97 mg, 0.960 mmol, 5 eq) were placed in a 2 dram vial containing 1.50 mL of methylene chloride. The reaction mixture was allowed to stir for 4 h, then filtered through a 300 mg silica gel cartridge. The cartridge was washed with an additional 1.0 mL EtOAc. The solvent was evaporated and the crude product was added to 0.74 mL of a 0.52M solution of the monosodium salt of urea in EtOH (0.384 mmol, 2 eq). The reaction mixture was heated to 80° C. with shaking for 19 h, and allowed to cool to RT. 1N HCl(aq), (0.40 mL) was added, and the crude product purified directly on prep HPLC. The solvent was removed from the appropriate fractions to obtain Example 10 (19.8 mg, 23%), as an off-white solid. (M−H=454.9).

### Examples 11-23

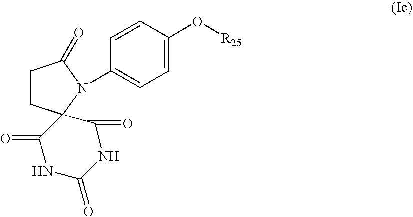

Compounds having the formula (Ic), wherein R₂₅ has the values listed in Table 1, were prepared following the same or similar procedure as for Example 6, using the appropriately selected arylboronic acid in place of 4-(methylthio)phenyl boronic acid in Step A.

| | | | | Other data |
|---|---|---|---|---|
| Ex. | | | MS (ESI)/ | (yield for 2 steps; |
| # | R₂₅ | Compound Name | (M-H) | characteristics) |
| 11 | 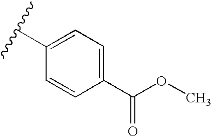 | 1-[4-(4-methoxycarbonyl-1- phenoxy)phenyl]-1,7,9- triazaspiro[4.5]decane-2,6,8,10- tetrone | 422 | 4.2 mg, 6.4%; cloudy film |
| 12 | 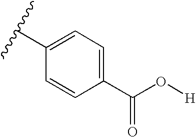 | 1-[4-(4-carboxy-1- phenoxy)phenyl]-1,7,9- triazaspiro[4.5]decane-2,6,8,10- tetrone | 407 | 7.4 mg, 11.6%; white solid |
| 13 | 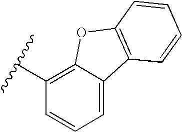 | 1-[4-(4-Dibenzo-1- furanoxy)phenyl]-1,7,9- triazaspiro[4.5]decane-2,6,8,10- tetrone | 454 | 1.2 mg, 1.7%; white solid |
| 14 | 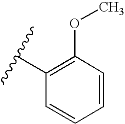 | 1-[4-(2-methoxy-1- phenoxy)phenyll-1,7,9- triazaspiro[4.5]decane-2,6,8,10- tetrone | 394 | 0.9 mg, 1.5%; white solid |
| 15 | 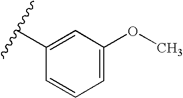 | 1-[4-(3-methoxy-1- phenoxy)phenyl]1,7,9- triazaspiro[4.5]decane-2,6,8,10- tetrone | 394 | 7.6 mg, 12.3%; white solid |
| 16 | 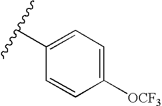 | 1-[4-(4-(Trifluoromethoxy)-1- phenoxy)phenyl]-1,7,9- triazaspiro[4.5]decane-2,6,8,10- tetrone | 448 | 5.8 mg, 8.3%; cloudy film |
| 17 | 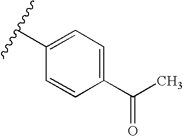 | 1-[4-(4-Acetyl-1- phenoxy)phenyl]-1,7,9- triazaspiro[4.5]decane-2,6,8,10- tetrone | 406 | 3.0 mg, 4.7%; cloudy film |
| 18 | 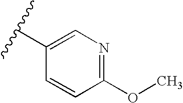 | 1-[4-(2-Methoxy-5- pyridineoxy)phenyl]-1,7,9- triazaspiro[4.5]decane-2,6,8,10- tetrone | 395 | 1.7 mg, 3.5%; cloudy film |
| 19 | 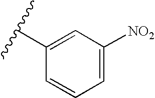 | 1-[4-(3-Nitro-1-phenoxy)phenyl]- 1,7,9-triazaspiro[4.5]decane- 2,6,8,10-tetrone | 409 | 0.4 mg, 0.8%; cloudy film |
| 20 | 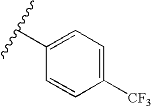 | 1-[4-(4-Trifluoromethyl-1- phenoxy)phenyl]-1,7,9- triazaspiro[4.5]decane-2,6,8,10- tetrone | 432 | 11.5 mg, 21.4%; cloudy film |
| 21 | 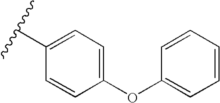 | 1-[4-(1,4-Diphenoxy)phenyl]- 1,7,9-triazaspiro[4.5]decane- 2,6,8,10-tetrone | 456 | 10.6 mg, 18.7%; cloudy film |
| 22 | 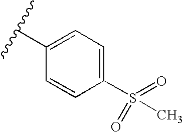 | 1-[4-(4-Methanesulfonyl-1- phenoxy)phenyl]-1,7,9- triazaspiro[4.5]decane-2,6,8,10- tetrone | 442 | 4 mg, 7.3%; cloudy film |
| 23 | 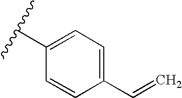 | 1-[4-(4-Ethenyl-1- phenoxy)phenyl]-1,7,9- triazaspiro[4.5]decane-2,6,8,10- tetrone | 390 | 5 mg, 10.3%; cloudy film |

### Example 24

1-[4-(2-Methyl-quinolin-4-ylmethoxy)-phenyl]-1,7,9-triaza-spiro[4.5]decane-2,6,8,10-tetraone

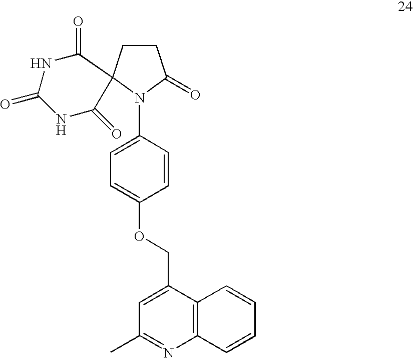

Step A: 1-(4-Benzyloxyphenyl)-1,7,9-triazaspiro[4.5]decane-2,6,8,10-tetrone

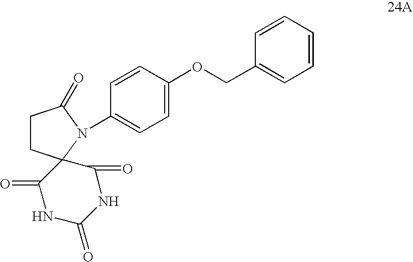

The same or similar procedure as in Example, Step B was followed to prepare example 3, step B was followed to prepare the intermediate N-(4-(Benzyloxy)phenyl)-5,5-diethyoxycarbonylpyrrolidone (6.63 g, 87%) from Compound 4A (6.64 g, 18.6 mmol), except after heating, the mixture was concentrated, dissolved in water (500 ml) extracted with DCM (3×250 ml), and the organic phase dried over Na2SO4, before concentration and purification by flash chromatography. The same or similar procedure as described in Example 1, Step C was then follows to cyclize the diethyoxycarbonyl intermediate to provide the tetrone as a white solid (57 mg, 46%). MS (ESI: 378 (M−H).

Step B: 1-(4-Hydroxyphenyl-1,7,9-triazaspiro[4,5]decane-2,6,8,10 tetrone

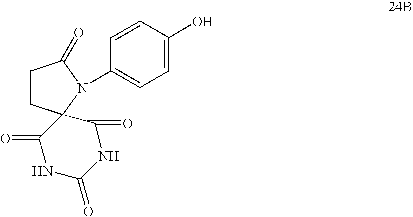

Palladium (5 mg, 10% on carbon) was added to 24A (20 mg, 0.053 mmol) and ammonium formate (55 mg, 1.2 mmol) in DMF (1 ml). The reaction mixture was heated at 40 C. for 20 minutes and filtered through a pad of silica gel. The filtrate was concentrated, diluted in EtOAc (50 ml) and washed with water (20 ml). The organic phase was dried over Na₂SO₄ and concentrated to give Example 24B as a white solid (4.8 mg, 32%). MS (ESI: 288 (M−H).

Step C: 1-[4-(2-methyl-quinolin-4-ylmethoxy)-phenyl]-5-oxo-pyrrolidine-2,2-dicarboxylic acid diethyl ester

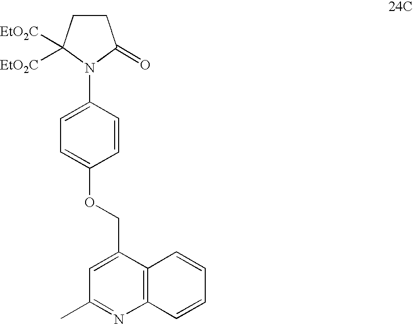

A mixture of phenol 24B (159 mg, 0.486 mmol), 4-chloromethyl-2-methylqulinoline (93 mg, 1.0 eq), Cs₂CO₃ (400 mg, 2.5 eq) and NaI (73 mg, 1.0 eq) in DMSO (2 ml) was stirred at rt for 2 h. The reaction was quenched with aqueous NH₄Cl, extracted with ethyl acetate, the combined organic extracts was dried and purified by flash column chromatography (100% ethyl acetate) to give (210 mg, 91.3%) as a colorless glass solid. MS Found: (M+H)⁺=477.

Step D: -[4-(2-Methyl-quinolin-4-ylmethoxy)-phenyl]-1,7,9-triaza-spiro[4.5]decane-2,6,8,10-tetraone

To a mixture of diethyl ester 24C (100 mg, 0.21 mmol) and urea (37.8 mg, 3 eq) in MeOH (2 ml) was added 25% NaOMe/MeOH solution (0.10 ml, 2.08 eq). The reaction mixture was stirred at 75° C. for 5 h, then cooled to rt, neutralized with 1N HCl solution mid extracted with CH₂Cl₂. The title compound, 24 (34.6 mg, 37%) was obtained after flash column chromatography (5% MeOH—CH₂Cl₂). MS Found: (M+H)⁺=445.

We claim:

1. A compound having the formula (I), 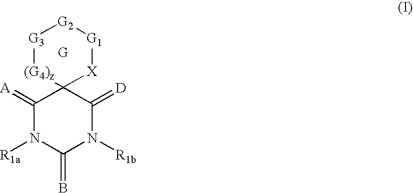
or a pharmaceutically-acceptable salt, hydrate, or prodrug thereof, wherein:
A, B and D are independently selected from oxygen and sulfur;
one of R₁ₐ and R_{1b} is hydrogen and the other of R₁ₐ and R_{1b} is selected from hydrogen, C₁₋₄alkyl, C₂₋₄alkenyl, and C₂₋₄alkynyl;
X is —NR₂—, —S—, —S(═O)—, or —S(O)₂—;
G₁ is —C(═O)—, —CR₃R₄—, —NR₁₁—, —CR₄═, or —N═;
G₂ is —O—, —C(═O)—, —CR₅R₆—, —NR₁₂—, —N═, or —CR₅═, except when G₁ is —CR₄═, or —N═, then G₂ is ═N— or ═CR₅—;
G₃ is —CR₇R₈—, —NR₁₃—, —N═, or —CR₇═, except when G₂ is —CR₅═ or —N═, then G₃ is ═CR₇— or ═N—;
G₄ is —CR₉R₁₀—, except when G₃ is —CR₇═ or —N═, then G₄ is ═CR₉—;
R₂ is Q-Ar, wherein Ar is substituted or unsubstituted aryl or substituted or unsubstituted heteroaryl, and Q is —C(═O)—, —CHR₁₄—, or a bond;
R₃, R₄, R₅, R₆, R₇, R₈, R₉, and R₁₀ are independently selected from the group consisting of hydrogen, halogen, nitro, cyano, alkyl, substituted alkyl, alkenyl, substituted alkenyl, hydroxy, alkoxy, phenyloxy, benzyloxy, amino, alkylamino, C(═O)H, acyl, CO₂H, alkoxycarbonyl, carbamyl, alkylthio, sulfonyl, sulfonamidyl, cycloalkyl, heterocycle, aryl, and heteroaryl, wherein each of R₃, R₄, R₅, R₆, R₇, R₈, R₉, and R₁₀ in turn is optionally substituted with one to two substituents selected from R₁₅;
R₁₁, R₁₂, and R₁₃ are independently selected from hydrogen, cyano, alkyl, substituted alkyl, alkenyl, substituted alkenyl, hydroxy, alkoxy, phenyloxy, benzyloxy, alkylamino, —C(═O)H, acyl, —CO₂H, alkoxycarbonyl, carbamyl, sulfonyl, sulfonamidyl, cycloalkyl, heterocycle, aryl, and heteroaryl, wherein each of R₁₁, R₁₂, and R₁₃ in turn is optionally substituted with one to two substituents selected from R₁₆;
R₁₄ is hydrogen, halogen, C₁₋₄alkyl, OH, OCH₃, or NH₂;
R₁₅ and R₁₆ are at each occurrence selected independently of each other from C₁₋₄alkyl, halogen, nitro, cyano, hydroxy, haloC₁₋₄alkyl, haloC₁₋₄alkoxy, amino, C₁₋₄alkylamino, C₁₋₄aminoalkyl, C₁₋₄hydroxyalkyl, C₃₋₇cycloalkyl, four to seven membered heterocyclo, five or six membered heteroaryl, phenyl, benzyl, phenyloxy, and benzyloxy; and
z is 0, 1, or 2 so that ring G is a four-to-seven membered spiroheterocyclo ring; provided that when Q is a bond,
(b) G₂ are selected from —O— and C(═O); or
(b) Ar is aryl or heteroaryl, each group optionally substituted with one to three of R₁₈ wherein:
R₁₈ is selected from alkyl, halogen, nitro, cyano, haloalkyl, haloalkoxy, hydroxy, alkoxy, (>C₁₀)aryl, (>C₈)cycloalkyl, (>C₁₀)heterocyclo, (>C₁₀)heteroaryl, A₁-NH-A₂-R₂₅, -A₁-O-A₂-R₂₆, -A₁-OC(═O)-A₂-R₂₅, -A₁-CO₂-A₂-R₂₅, -A₁-NR₁₉C(═O)-A₂-R₂₅, -A₁-NR₁₉C(═O)NR₂₀-A₂-R₂₅, -A₁-NR₁₉CO₂-A₂-R₂₅, -A₁-NR₁₉SO₂NR₂₀-A₂-R₂₅, -A₁-C(═O)NR₁₉-A₂-R₂₅, -A₃-O-A₂, A₃-S-A₂, -A₃-SO₂-A₂-, -A₃-NR₁₉-A₂-, -A₄-C(═O)-A₅-, A₄-S(═O)-A₅-, -A₄-NR₁₉SO₂-A₅-, and -A₄-SO₂NR₁₉-A₅-,
A₁ is —(CR₂₁R₂₂)ᵣ—;
A₂ is —(CR₂₃R₂₄)ₛ—;
A₃ is —(CR₂₁R₂₂)ₜ—;
A₄ is —(CR₂₁R₂₂)ᵤ—;
A₅ is —(CR₂₃R₂₄)ᵥ—;
r and s are selected from 0, 1, 2, 3, and 4;
t is 2, 3 or 4;
u and v are 0-4 provided that u and v are not both 0;
R₁₉, R₂₀, R₂₁, R₂₂, R₂₃, and R₂₄ are selected from hydrogen, C₁₋₄alkyl, hydroxyC₁₋₄alkyl, haloC₁₋₄alkyl, amino, and aminoC₁₋₄alkyl; and
R₂₅ is selected from hydrogen, C₁₋₆alkyl, amino, C₁₋₆alkylamino, aryl, cycloalkyl, heterocyclo, and heteroaryl, each group optionally substituted with R₂₇ and/or R₂₈;
R₂₆ is
(c) diphenoxy, (>C₈)cycloalkyl, (>C₁₀)heterocyclo, or (>C₁₀)heteroaryl, each group optionally substituted with R₂₇ and/or R₂₈; or
(d) aryl substituted with —SC₁₋₄alkyl, —OC₁₋₄alkyl, —SC₁₋₄haloalkyl, —OC₁₋₄haloalkyl, nitro, haloalkyl, C₂₋₄alkenyl, —CO₂H, —CO₂C₁₋₄alkyl, —SO₂C₁₋₄alkyl or —C(═O) C₁₋₄alkyl; and
R₂₇ and R₂₈ are independently selected from C₁₋₄alkyl, C₂₋₄alkenyl, hydroxy, —OC₁₋₄alkyl, halogen, cyano, nitro, —CF₃, —OC₁₋₄haloalkyl, —SC₁₋₄alkyl, —SO₂C₁₋₄alkyl, —CO₂H, —CO₂C₁₋₄alkyl, —C(═O) C₁₋₄alkyl, phenyloxy, and benzyloxy.

2. A compound according to claim 1, or a pharmaceutically-acceptable salt, hydrate, or prodrug thereof, having the formula (Ia) 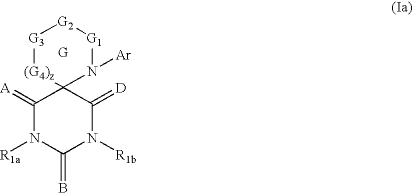
or a pharmaceutically-acceptable salt, hydrate, or prodrug thereof, wherein:
one of R₁ₐ and R_{1b} is hydrogen and the other of R₁ₐ and R_{1b} is selected from hydrogen, C₁₋₄alkyl, C₂₋₄alkenyl, and C₂₋₄alkynyl;
X is —NR₂—, —S—, —S(═O)—, or —S(O)₂—;
G₁ is —C(═O)—, —CR₃R₄—, —NR₁₁—, —CR₄═, or —N═;
G₂ is —O—, —C(═O)—, —CR₅R₆—, —NR₁₂—, —N═, or —CR₅═, except when G₁ is —CR4═, or —N═, then G₂ is ═N— or ═CR₅—;
G₃ is —CR₇R₈—, —NR₁₃—, —N═, or —CR₇═, except when G₂ is —CR₅═ or —N═, then G₃ is ═CR₇— or ═N—;
G₄ is —CR₉R₁₀—, except when G₃ is —CR₇═ or —N═, then G₄ is ═CR₉—;
Ar is aryl or heteroaryl, each group optionally substituted with one to two R₁₈;
R₃, R₄, R₅, R₆, R₇, R₈, R₉, and R₁₀ are independently selected from the group consisting of hydrogen, halogen, nitro, cyano, C₁₋₄alkyl, hydroxy, trifluoromethyl, trifluoromethoxy, N-phenyloxy, benzyloxy, alkylamino, C(═O)H, CO₂H, C(═O) (C₁₋₄alkyl), and CO₂(C₁₋₄alkyl);
R₁₁ and R₁₂ are independently hydrogen or C₁₋₄alkyl;
R₁₈ is selected from alkyl, halogen, nitro, cyano, haloalkyl, haloalkoxy, hydroxy, alkoxy, (>C₁₀)aryl, (>C₈)cycloalkyl, (>C₁₀)heterocyclo, (>C₁₀)heteroaryl, A₁-NH-A₂-R₂₅, -A₁-O-A₂-R₂₆, -A₁-OC(═O)-A₂-R₂₅, -A₁-CO₂-A₂-R₂₅, -A₁-NR₁₉C(═O)-A₂-R₂₅, -A₁-NR₁₉C(═O)NR₂₀-A₂-R₂₅, -A₁-NR₁₉CO₂-A₂-R₂₅, -A₁-NR₁₉SO₂NR₂₀-A₂-R₂₅, -A₁-C(═O)NR₁₉-A₂-R₂₅, -A₃-O-A₂, A₃-S-A₂, -A₃-SO₂-A₂-, -A₃-NR₁₉-A₂-, -A₄-C(═O)-A₅-, A₄-S(═O)-A₅-, -A4-NR₁₉SO₂-A₅-, and -A₄-SO₂NR₁₉-A₅-,
A₁ is —(CR₂₁R₂₂)ᵣ—;
A₂ is —(CR₂₃R₂₄)ₛ—;
A₃ is —(CR₂₁R₂₂)ₜ—;
A₄ is —(CR₂₁R₂₂)ᵤ—;
A₅ is —(CR₂₃R₂₄)ᵥ—;
r and s are selected from 0, 1,2,3, and 4;
t is 2, 3 or 4;
u and v are 0-4 provided that u and v are not both 0;
R₁₉, R₂₀, R₂₁, R₂₂, R₂₃, and R₂₄ are selected from hydrogen, C₁₋₄alkyl, hydroxyC₁₄alkyl, haloC₁₋₄alkyl, amino, and aminoC₁₋₄alkyl;
R₂₅ is selected from hydrogen, C₁₋₆alkyl, amino, C₁₋₆alkylamino, aryl, cycloalkyl, heterocyclo, and heteroaryl, each group optionally substituted with R₂₇ and/or R₂₈;
R₂₆ is
(a) diphenoxy, (>C₈)cycloalkyl, (>C₁₀)heterocyclo, or (>C₁₀)heteroaryl, each group optionally substituted with R₂₇ and/or R₂₈;or
(b) aryl substituted with —SC₁₋₄alkyl, —OC₁₋₄alkyl, —SC₁₋₄haloalkyl, —OC₁₋₄haloalkyl, nitro, haloalkyl, C₂₋₄alkenyl, —CO₂H, —CO₂C₁₋₄alkyl, —SO₂C₁₋₄alkyl or —C(═O) C₁₋₄alkyl; and
R₂₇ and R₂₈ are independently selected from C₁₋₄alkyl, C₂₋₄alkenyl, hydroxy, —OC₁₋₄alkyl, halogen, cyano, nitro, —CF₃, —OC₁₋₄haloalkyl, —SC₁₋₄alkyl, —SO₂C₁₋₄alkyl, —CO₂H, —CO₂C₁₋₄alkyl, —C(═O) C₁₋₄alkyl, phenyloxy, and benzyloxy; and
z is 0, or 1 so that ring G is a five to six-membered spiroheterocyclo ring.

3. A compound according to claim 2 or a pharmaceutically-acceptable salt, hydrate, or prodrug thereof, having the formula (Ib), 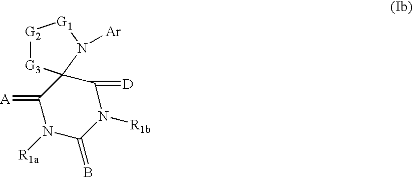
or a pharmaceutically-acceptable salt, hydrate or prodrug thereof, in which:
one of G₁ and G₂ is CR₅R₆, and the other of G₁ and G₂ is —C(═O)—;
G₃ is —CR₇R₈;
R₅, R₆, R₇ and R₈ are independently H or C₁₋₄alkyl; and
Ar is phenyl, pyridyl, pyrazinyl, pyrimidinyl, or napthyl, each of which is optionally substituted with R₁₈.
R₁₈ is selected from C₁₋₄alkyl, halogen, hydroxy, C₁₋₄alkyoxy, A₁-NH-A₂-R₂₅, -O-A₁-NHC(═O)R₂₅, -O-R₂₆, or (>C10)heteroaryl;
R₂₅ is selected from hydrogen, C₁₋₆alkyl, phenyl, pyridyl, indolyl, napthyl, and 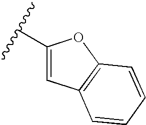
each group optionally substituted with R₂₇ and/or R₂₈ where valence allows;
R₂₆ is
(a) diphenoxy or (>C₁₀)heteroaryl, each group optionally substituted with R₂₇ and/or R₂₈; or
(b) phenyl substituted with —SCH₃, —O CH₃, —S CF₃, —OC₁₋₄ CF₃, nitro, CF₃, C₂alkenyl, —CO₂H, —CO₂ CH₃, —SO₂CH₃ or —C(═O)CH₃; and
R₂₇ and R₂₈ are independently selected from C₁₋₄alkyl, C₂₋₄alkenyl, hydroxy, —OC₁₋₄alkyl, halogen, cyano, nitro, —CF₃, —OCF₃, —SCH₃, —SO₂CH₃, —CO₂H, —CO₂CH₃, —C(═O)CH₃, phenyloxy, and benzyloxy.

4. A compound according to claim 3 wherein:
G₁ is —C(═O)—;
G₂ is CR₅R₆;
G₃ is —CR₇R₈;
R₅ is H or; C₁₋₄alkyl;
R₆, R₇, and R₈ are hydrogen;
Ar is phenyl optionally substituted in the para position by R₁₈;
R₁₈ is selected from C₁₋₄alkyl, bromo, hydroxy, methoxy, A₁-NH-A₂-R₂₅, —O-A₁-NHC(═O)R₂₅, and —O-R₂₆;
R₂₅ is selected from hydrogen, C₁₋₆alkyl, phenyl, pyridyl, indolyl, napthyl and 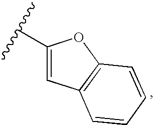
each group optionally substituted with R₂₇ and/or R₂₈ where valence allows; and
R₂₆ is selected from.

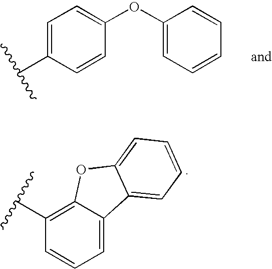

5. A compound according to claim 1, or a pharmaceutically-acceptable salt, hydrate or prodrug thereof, in which R₁ₐ and R_{1b} are both hydrogen.

6. A compound according to claim 1, or a pharmaceutically-acceptable salt, hydrate or prodrug thereof, in which A, B and D are all oxygen.

7. A compound according to claim 3, or a pharmaceutically-acceptable salt, hydrate or prodrug thereof,
wherein:
G₁ is —C(═O)—;
G₂ is —C(C₁₋₄alkyl)(H)—;
G₃ is —CH₂—; and
R₂₆ is phenyl substituted with —SCH₃, —O CH₃, —S CF₃, —OC₁₋₄CF₃, nitro, CF₃, C₂alkenyl, —CO₂H, —CO₂ CH₃, —SO₂ CH₃ or —C(═O)CH₃.

8. A compound of claim I selected from:
(i) 1-(4-Bromophenyl)-1,7,9-triazaspiro[4,5]decane-2,6,8,10-tetrone;
1-Phenyl-1,7,9-triazaspiro[4,5]decane-2,6,8,10-tetrone;
1-[4-(1,4-biphenyl)phenyl]-1,7,9-triazaspiro[4,5]decane-2,6,8,10-tetrone;
1-(4-Methoxyphenyl)-1,7,9-triazaspiro[4,5]decane-2,6,8,10-tetrone;
1-(4-Hydroxyphenyl)-1,7,9-triazaspiro[4,5]decane-2,6,8,10-tetrone;
1-[4-(4-(methylthio)-1-phenoxy)phenyl]-1,7,9-triazaspiro[4.5]decane-2,6,8,10-tetrone;
3-(R,S)-Methyl-1-(1-phenoxyphenyl)-1,7,9-triazaspiro[4,5]decane-2,6,8,10-tetrone;
4-(R)-Ethyl-1-(1-phenoxyphenyl)-1,7,9-triazaspiro[4,5]decane-2,6,8,10-tetrone;
4-(S)-Ethyl-1-(1-phenoxyphenyl)-1,7,9-triazaspiro[4,5]decane-2,6,8,10-tetrone;
1-(4-(((((Benzofuran-2-yl)carbonyl)amino)ethyl)oxy)phenyl)-1,7,9-triazaspiro[4,5]decane-2,6,8,10-tetrone;
1-[4-(((4-Phenyloxy)phenyl)amino)phenyl]-1,7,9-triazaspiro[4.5]decane-2,6,8,10-tetrone;
1-[4-(4-methoxycarbonyl-1-phenoxy)phenyl]-1,7,9-triazaspiro[4.5]decane-2,6,8,10-tetrone;
1-[4-(4-carboxy-1-phenoxy)phenyl]-1,7,9-triazaspiro[4.5]decane-2,6,8,10-tetrone;
1-[4-(4-Dibenzo-1-furanoxy)phenyl]-1,7,9-triazaspiro[4.5]decane-2,6,8,10-tetrone;
1-[4-(2-methoxy-1-phenoxy)phenyl]-1,7,9-triazaspiro[4.5]decane-2,6,8,10-tetrone;
1-[4-(3-methoxy-1-phenoxy)phenyl]-1,7,9-triazaspiro[4.5]decane-2,6,8,10-tetrone;
1-[4-(4-(Trifluoromethoxy)-1-phenoxy)phenyl]-1,7,9-triazaspiro[4.5]decane-2,6,8,10-tetrone;
1-[4-(4-Acetyl-1-phenoxy)phenyl]-1,7,9-triazaspiro[4.5]decane-2,6,8,10-tetrone;
1-[4-(2-Methoxy-5-pyridineoxy)phenyl]-1,7,9-triazaspiro[4.5]decane-2,6,8,10-tetrone;
1-[4-(3-Nitro-1-phenoxy)phenyl]-1,7,9-triazaspiro[4.5]decane-2,6,8,10-tetrone;
1-[4-(4-Trifluoromethyl-1-phenoxy)phenyl]-1,7,9-triazaspiro[4.5]decane-2,6,8,10-tetrone;
1-[4-(1,4-Diphenoxy)phenyl]-1,7,9-triazaspiro[4.5]decane-2,6,8,10-tetrone;
1-[4-(4-Methanesulfonyl-1-phenoxy)phenyl]-1,7,9-triazaspiro[4.5]decane-2,6,8,10-tetrone; and
1-[4-(4-Ethenyl-1-phenoxy)phenyl]-1,7,9-triazaspiro [4.5]decane-2,6,8,10-tetrone; or
(ii) a pharmaceutically-acceptable salt, hydrate or prodrug of said compound.

9. A compound according to claim 1, or a pharmaceutically-acceptable salt, hydrate or prodrug thereof, in which R1a and R1b are both hydrogen.

10. A compound according to claim 1 wherein ring G is selected from: 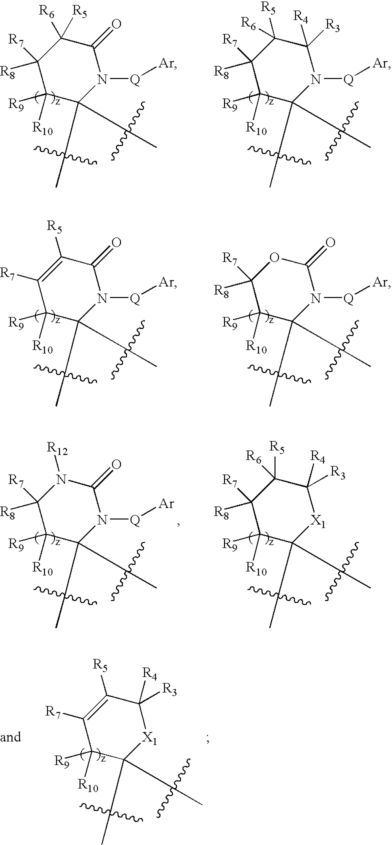
X₁ is selected from —S—, —S(═O)—, and —S(O)₂—;
Q is —CHR₁₄—, or C(═O);
Ar is aryl or heteroaryl optionally substituted with one to three R₁₈;
R₃, R₄, R₅, R₆, R₇, R₈, R₉, and R₁₀ are independently selected from the group consisting of hydrogen, halogen, nitro, cyano, amino, C₁₋₄alkyl, C₂₋₄alkenyl, hydroxy, C₁₋₄alkoxy, phenyloxy, benzyloxy, C₁₋₄alkylamino, C₁₋₄aminoalkyl, C₁₋₄hydroxyalkyl, C₃₋₇cycloalkyl, four to seven membered heterocyclo, five or six membered heteroaryl, phenyl, benzyl, phenyloxy, and benzyloxy;
R₁₄ is hydrogen, halogen, C₁₋₄alkyl, OH, OCH₃, or NH₂;
r and s are independently 0, 1, or 2; and
z is 0 or 1.

11. A pharmaceutical composition comprising at least one compound of claim 1, or a salt, hydrate, or prodrug thereof, and a pharmaceutically-acceptable vehicle or carrier.

12. A pharmaceutical composition comprising at least one compound of claim 3, or a salt, hydrate, or prodrug thereof, and a pharmaceutically-acceptable vehicle or carrier.

13. The pharmaceutical composition of claim 12 further comprising at least one other therapeutic agent selected from anti-inflammatory agents, anti-viral agents, immunosuppressants, antiproliferative agents, antitumor agents, and/or TNF-α inhibitors.

14. A method of treating a MMP-13 associated disorder comprising administering an effective amount of at least one compound of claim 1, or a pharmaceutically-acceptable salt, prodrug, or hydrate thereof, to a patient in need thereof.

15. The method of claim 14 wherein the MMP-13-associated disorder is selected from osteoarthritis and rheumatoid arthritis.
